(19) United States
(12) Patent Application Publication
(10) Pub. No.: US 2014/0308241 A1
(43) Pub. Date: Oct. 16, 2014

(54) BIOMARKERS FOR T CELL MALIGNANCIES AND USES THEREOF

(71) Applicants: British Columbia Cancer Agency Branch, Vancouver (CA); The University of British Columbia, Vancouver (CA)

(72) Inventors: Youwen Zhou, Vancouver (CA); Yuanshen Huang, Vancouver (CA); Yang Wang, Beijing (CN); Ming-wan Su, Vancouver (CA)

(21) Appl. No.: 14/358,869

(22) PCT Filed: Nov. 16, 2012
(86) PCT No.: PCT/CA2012/001052

§ 371 Date: May 16, 2014

Related U.S. Application Data
(60) Provisional application No. 61/560,745, filed on Nov. 16, 2011.

(51) Int. Cl. C12Q 1/68

(52) U.S. Cl. CPC C12Q 1/6886
USPC 424/85.2; 506/9; 514/567; 514/616; 514/431; 514/569

(57) ABSTRACT

Described are biomarkers including TOX useful for the diagnosis or prognosis of T cell malignancy. A level of a biomarker is determined in a sample from a subject and compared to a control level, wherein an increased level of the biomarker in the sample relative to the control level indicates that the subject has T cell malignancy. The T cell malignancy may be a cutaneous T cell lymphoma (CTCL) such as mycosis fungoides or Sezary syndrome.

### CROSS REFERENCE TO RELATED APPLICATIONS

This application claims priority to U.S. Provisional Patent Application No. 61/560,745 filed on Nov. 16, 2011, which is hereby incorporated by reference in its entirety.

### FIELD OF THE DISCLOSURE

The disclosure relates to biomarkers for T cell malignancies and more specifically to diagnostic and prognostic biomarkers and associated methods for T cell malignancies such as mycosis fungoides and Sezary syndrome.

### BACKGROUND OF THE DISCLOSURE

There are a group of T cell derived malignancies affecting humans, including cutaneous T cell lymphomas (CTCL), peripheral T cell lymphomas, T cell leukemias, and their histological and clinical variants. Although the combined overall incidence of CTCL is low, at less than 10 per million population per year, they are often difficult to differentiate from far more common disease conditions, such as chronic dermatitis (approximately 10% of the general population), cutaneous reaction to drugs (1-5% of population), psoriasis (1.5% of population) and pityriasis rubra pilaris (approximately 0.1% of population), especially at an early stage of disease. The primary method of diagnosis is by clinical suspicion, histological criteria on skin biopsies, flow-cytometry based immune-phenotyping of the blood cells when they are present, and by analysis of the T cell receptor gene rearrangement status. In histological and flow cytometry analyses, negative “markers” are often used to aid the diagnosis, including loss of CD7, CD2, CD3, CD28, and so on, however, none are very specific. There are no specific positive diagnostic markers for these T cell malignancies so far.

In the case of Sezary syndrome, a leukemic variant of CTCL, the cancerous cells are much larger and have cerebriform nucleus, and often have loss of CD7 (but not always).

Although a diagnosis is rarely established for clinically suspected cases, many cases are delayed, sometimes by as long as 10 years, even after repeated serial biopsies. Therefore, establishing a diagnosis of CTCL is one of the major diagnostic challenges for any pathology laboratory worldwide. A specific diagnostic and prognostic marker will be frequently used to rule out CTCL for common diseases such as chronic dermatitis, drug reactions, and psoriasis.

The diagnosis of early mycosis fungoides (eMF, patch and early plaque mycosis fungoides (Pimpinelli, Olsen et al. 2005)) has been a major diagnostic challenge in dermatology. The difficulty arises because of the lack of specific cellular or molecular markers that can reliably differentiate the malignant T cells from the abundant reactive T cells that are present not only in the eMF lesions themselves, but also in the benign inflammatory mimickers of eMF. Because of the lack of sensitive and specific histologic markers, it takes months to even decades before a conclusive diagnosis of MF can be made in many clinical cases (Arai, Katayama et al. 1991). The lack of a standardized and reliable method for diagnosing MF presents significant difficulties in the assessment and management of patients suspected to have MF, in the development and evaluation of therapies, and in establishing a long term prognosis for patients. Recognizing this difficulty, and in an attempt to establish a standardized algorithm for making the diagnosis of eMF, the International Society of Cutaneous Lymphomas (ISCL) proposed an integrated clinical pathological algorithm for diagnosing eMF (Olsen, Vonderheid et al. 2007). While this has been accepted by many as a useful diagnostic system, clinical experience with this system will be needed over a long period of time to fully evaluate its clinical utility. In addition, further modifications of this system have been proposed by Ferrara et al (Ferrara, Di Blasi et al. 2008). It is of note that the molecular markers and immunohistochemistry markers considered as ancillary diagnostic criteria by the ISCL are all negative markers: MF skin biopsies are characterized by the loss of expression of cellular and molecular markers such as CD7, CD2, CD3, and CD28. Positive molecular markers for defining MF in general, and eMF in particular, are lacking.

The lack of a specific and reliable marker differentiating early mycosis fungoides (eMF) from benign inflammatory dermatitis presents significant difficulties in the assessment and management of patients suspected to have MF, which often leads to delayed conclusive diagnosis and improper medical care approaches.

There remains a need for biomarkers useful for the diagnosis and prognosis of T cell malignancies.

### SUMMARY OF THE DISCLOSURE

The inventors have determined that the biomarkers listed in Table 2 are useful for identifying subjects with T cell malignancies. The biomarkers listed in Table 2 were identified as differentially expressed in subjects with early mycosis fungoides (eMF) relative to subjects with chronic dermatitis or normal skin. Subjects with cutaneous T cell lymphoma (CTCL) may present with symptoms similar to benign inflammatory dermatoses such as chronic dermatitis, hampering the diagnosis of more serious malignant disease. Biomarkers that are differentially expressed in T cell malignancies are therefore particularly useful for diagnosing or detecting T cell malignancies.

In a preferred embodiment, it has also been determined that TOX is useful as a diagnostic and prognostic biomarker for T cell malignancies such as CTCL. Expression of TOX has been shown to correlate with the severity of disease in subjects with CTCL and is also useful for predicting mortality in subjects with the disease. Increases in the level of TOX have been shown to parallel the progression of mycosis fungoides in subjects with stage I to stage IV disease. Biopsies from subjects with eMF also showed highly specific staining for TOX using immunohistochemistry and immunofluorescence. T-lineage acute lymphoblastic leukemia cell lines were also shown to express TOX indicating that TOX is useful as a biomarker in non-CTCL T cell malignancies.

Accordingly, in one aspect there is provided a method of screening for, diagnosing or detecting T cell malignancy in a subject, the method comprising:
(a) determining a level of one or more biomarkers listed in Table 2 in a sample from the subject; and
(b) comparing the level of the one or more biomarkers in the sample to a control level, wherein an increased level of the one or more biomarkers in the sample relative to the control level indicates that the subject has T cell malignancy.

In one embodiment, the biomarker is TOX. In some embodiments, the T cell malignancy is cutaneous T cell Lymphoma (CTCL), peripheral T cell lymphoma or T cell leukemia. In some embodiments, the CTCL is mycosis fungoides (MF), early mycosis fungoides (eMF) or Sezary syndrome. In one embodiment, the control level is representative of the level of a biomarker in subjects without T cell malignancy. In some embodiments, the methods described herein include determining a level of one or more biomarkers selected from CD7, CD2, CD3 and CD28, wherein the absence or a reduced level of CD7, CD2, CD3 or CD28 relative to a control indicates that the subject has T cell malignancy. In one embodiment, the method includes determining a level of one or more of the biomarkers listed in Table 2 and one or more biomarkers selected from CD7, CD2, CD3 and CD28. In one embodiment, the methods described herein include determining a level of TOX and a level of CD7 in a sample from a subject and comparing the level of TOX and the level of CD7 to a control level of TOX and a control level of CD7 wherein an increased level of TOX and a decreased level of CD7 in the sample indicates that the subject has T cell malignancy. In some embodiments, the method includes contacting the sample with a detection agent for a biomarker, such as a detection agent for TOX. In some embodiments, the method further comprises treating a subject identified as having a T cell malignancy for the disease.

In one aspect, there is provided a method of monitoring T cell malignancy in a subject comprising:
(a) determining a level of TOX in a sample from the subject at a first time point;
(b) determining a level of TOX in a sample from the subject at a second time point and comparing the level of TOX in the sample at the first time point with the level of TOX in the sample at the second time point.

In one embodiment, an increase in the level of TOX is indicative of an increase in severity of T cell malignant disease and a decrease in the level of TOX is indicative of a decrease in severity of disease. In one embodiment, the magnitude of the increase or decrease is indicative of the magnitude of the change in severity of the disease. In some embodiments, the T cell malignancy is cutaneous T cell lymphoma (CTCL), peripheral T cell lymphoma or T cell leukemia. In some embodiments, the CTCL is mycosis fungoides (MF), early mycosis fungoides (eMF) or Sezary syndrome. In some embodiments, the method includes contacting the sample with a detection agent for a biomarker, such as a detection agent for TOX.

In one aspect, there is provided a method of providing a prognosis for a subject with T cell malignancy comprising:
(a) determining a level of TOX in a sample from the subject; and
(b) comparing the level of TOX in the sample to a control level.

In one embodiment, the control level is representative of a level of TOX in one or more samples from subjects without T cell malignancy, such as samples of normal skin or samples from subjects with benign inflammatory dermatoses. In one embodiment, the control level is representative of a level of TOX in one or more samples from subjects with T cell malignancy, wherein the severity or outcome of the disease is known. For example, in one embodiment the control level is representative of a level of TOX in one or more samples from subjects with stage I, stage II, stage III or stage IV disease. In one embodiment, magnitude of the level of TOX in the sample relative to the control level is indicative of the severity of the disease. In some embodiments, the T cell malignancy is cutaneous T cell lymphoma (CTCL), peripheral T cell lymphoma or T cell leukemia. In some embodiments, the CTCL is mycosis fungoides (MF), early mycosis fungoides (eMF) or Sezary syndrome. In some embodiments, the method includes contacting the sample with a detection agent for a biomarker, such as a detection agent for TOX.

In some aspects, the methods described herein include obtaining one or more samples from a subject at one or more time points. In some embodiments, the sample is a tissue sample or blood sample. In one embodiment, the sample comprises CD4+ T cells. In some aspects, the methods described herein include testing the sample for the expression of one or more biomarkers listed in Table 2. In some embodiments, the methods described herein include testing the sample for the expression of one or more biomarkers by contacting the sample with a detection agent, such as an antibody or nucleic acid. In one embodiment, the biomarker is TOX. In one embodiment, the methods described herein include detecting and optionally quantifying the detection agent. In some embodiments, the methods described herein further comprise treating a subject identified as having a T cell malignancy for the disease or making treatment decisions based on the level of TOX in a sample from the subject. In one embodiment, the methods further comprise administering an anticancer therapy or antineoplastic agent to a subject identified as having a T cell malignancy based on the level of TOX in a sample from the subject.

In another aspect, there is provided a kit comprising one or more reagents for conducting a method according to a method described herein. In some embodiments, the kit includes instructions for use and/or containers suitable for containing one or more of the reagents. In one embodiment, the reagents include a detection agent for detecting a biomarker listed in Table 2. In one embodiment, the kit includes a detection agent for detecting TOX. In one embodiment, the detection agent is an antibody that selectively binds the TOX protein. In one embodiment, the detection agent is a nucleic acid that selectively binds a nucleic acid that codes for the TOX protein, such as a nucleic acid probe or a primer suitable for amplifying all or part of a nucleic acid that codes for the TOX protein.

Other features and advantages of the present disclosure will become apparent from the following detailed description. It should be understood, however, that the detailed description and the specific examples while indicating preferred embodiments of the disclosure are given by way of illustration only, since various changes and modifications within the spirit and scope of the disclosure will become apparent to those skilled in the art from this detailed description.

### BRIEF DESCRIPTION OF THE DRAWINGS

One or more embodiments of the disclosure will now be described in relation to the drawings in which:

FIG. 1 shows the identification of eMF specific genes. Panel A: Comparative transcriptome analyses of eMF were performed using Agilent G4112F whole human genome arrays as described in the text. The transcripts with >2 fold differential expression between eMF and normal skin (NS) are depicted as red dots in the volcano plot using GeneSpring software (version 7.3). Line “a” represents the threshold of p values <0.05 without correction. Line “b” represents the threshold of p<0.05, after Bonferroni correction for multiple testing. Panel B: The 439 transcripts differentially expressed in eMF relative to NS are plotted as a heat map in a dataset consisting of 25 transcriptomes analyzed, including 5 eMF, 5 benign inflammatory dermatosis or BID (all 5 were chronic dermatitis, or CD); and 15 NS. Panel C: A heat map showing the 19 genes with significant up-regulation in eMF (>2 fold) but not in BID (<2 fold) when compared with NS. Panels D and E: Quantification of TOX and PDCD1 transcripts (respectively) in eMF (N=21), BID (N=15, including 6 CD, 6 psoriasis and 3 pityriasis rubra pilaris), and NS (N=21) using RT-PCR. The relative transcript levels are expressed as copies of TOX or PDCD1 per 1000 copies of glyceraldehydes phosphate dehydrogenase (GAPDH) transcripts.

FIG. 2 shows staining of TOX protein in eMF and CD. Panel A: Frozen sections (4 um) of eMF and CD biopsies were stained with a specific rabbit polyclonal antibody against TOX protein (Red) and a mouse monoclonal antibody against CD4 antigen (Green) using multi-colored immunofluorescence protocol. DAPI stain was used to visualize the nuclei of cells. Panel B: Punch biopsies of lesional skin were obtained from patients with eMF and CD and immediately frozen in OCT at −80° C. (Tissue-Tek®; Sakura Finetek, Torrance, Calif., USA). The biopsies were then cut with a cryostat into 4 um thick sections for immunohistochemistry analysis. Briefly, the sections were fixed in 4% paraformaldehyde at 4° C. for 20 minutes and subjected to standard immunohistochemistry protocol using the Vector Elite ABC kit (Vector Laboratories, Inc., Burlingame, Calif., USA). The sections were incubated in polyclonal anti-TOX (Sigma-Aldrich, Oakville, ON, CA). The stained samples were viewed under a Motic light microscope (Motic, Richmond, BC, Canada). Top panels: Patch stage MF. Shown are epidermotropic MF cells in a Pautrier's microabscess and papillary dermal MF cells staining positive with TOX antibody. Magnification: 400×. Bottom panels: Chronic dermatitis; No significant TOX staining in the epidermis, or in the dermis.

FIG. 3 shows staining of TOX, CD8, CD1a and PDCD1 in eMF and CD. Frozen sections (4 um) of eMF and CD biopsies were stained with a specific rabbit polyclonal antibody against TOX protein (Red) and a mouse monoclonal antibody against CD8 (Panel A, Green), CD1a (Panel B, green) or PDCD1 (Panel C, green). To visualize the nucleus of cells, DAPI counter-stain was also performed (blue). (Magnification: 400×). eMF: Early mycosis fungoides; CD: chronic dermatitis.

FIG. 4 shows differentially expressed genes in late Stage CTCL. The late CTCL cells were purified from patients with Sezary syndrome (N=6) using CD4+CD7− as a guide using negative purification with magnetic purification kits (StemCell Technologies, Vancouver, BC). The control cells were also purified using similar kits except focusing on the CD4+CD7+ cell population in volunteers with no CTCL (N=9). The analysis of differential gene expression for late CTCL cells is the same as described above for FIG. 1 Panel A). Highlighted is the only gene (TOX) that is up-regulated in both early CTCL samples (4 fold, p=0.02) and late CTCL samples (8 fold, p=0.001).

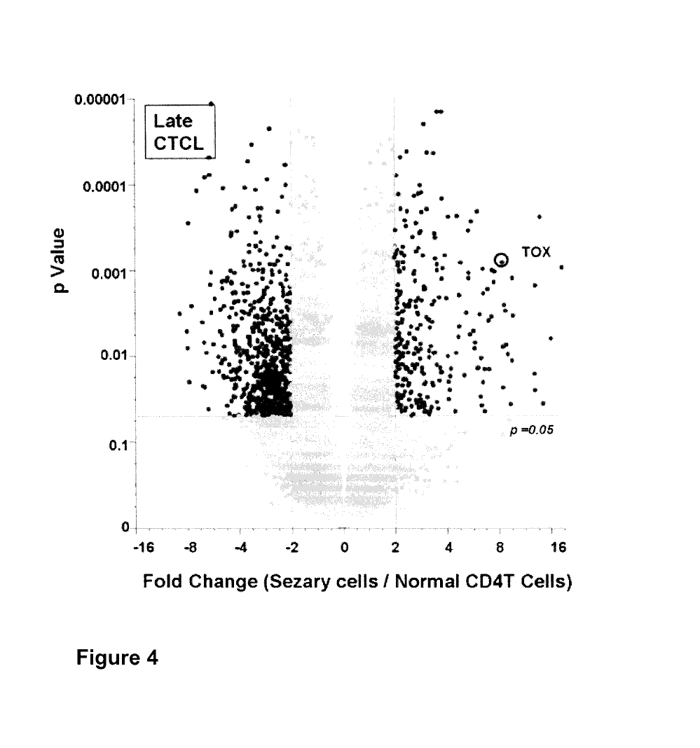

FIG. 5 shows that MF tissues contain higher TOX mRNA level, compared with control skin tissues. TOX mRNA is quantified using real-time quantitative polymerase chain reaction in mycosis fungoides (MF, N=123), benign inflammatory dermatoses (BID, N=22) including psoriasis, chronic dermatitis and pityriasis rubra pilaris), or normal skin (NS, N=6) biopsies. The expression levels were normalized to beta actin mRNA levels. (TOX mRNA per 1000 copies of beta actin mRNA). Horizontal bars denote the average and standard deviation for each skin type analyzed. None MF denoted BID and NS combined.

FIG. 6 shows that the increase of TOX mRNA level parallels the disease progression of MF, from stage I to stage IV. TOX mRNA is quantified using real-time quantitative polymerase chain reaction in mycosis fungoides (MF, N=123), benign inflammatory dermatoses (BID, N=22, including psoriasis, (PSO, N=7) chronic dermatitis (BCD, N=11) and pityriasis rubra pilaris (PRP, N=3)) or normal skin (NS, N=6) biopsies. As in FIG. 5, the expression levels were stratified accordingly to disease stage and normalized to beta actin mRNA levels.

FIG. 7 shows ROC analysis of TOX mRNA level as a marker for MF. ROC analysis was performed on mRNA, which is quantified using real-time quantitative polymerase chain reaction in mycosis fungoides (MF, N=123), benign inflammatory dermatoses (BID, N=22, including psoriasis, chronic dermatitis and pityriasis rubra pilaris) or normal skin (NS, N=6) biopsies. The expression levels were normalized to beta actin mRNA levels. (TOX mRNA per 1000 copies of beta actin mRNA). Horizontal bars denote the average and standard deviation for each skin type analyzed.

FIG. 8 shows progression risk according to TOX mRNA levels in MF (entire patient population). The MF skin biopsies were divided to the TOX high group (TOX level higher than the top sample of the control group) and the TOX low group (level no different than the benign inflammatory dermatoses). The five year occurrence of progressing to at least 1 numerical grade higher are shown in all MF patients analyzed (N=77).

FIG. 9 shows progression risk according to TOX mRNA levels in MF (only patients with early stage-patch or plaque disease). The MF skin biopsies were divided to the TOX high group (TOX level higher than the top sample of the control group) and the TOX low group (level no different than the benign dermatoses). The five year occurrence of progressing to at least 1 numerical grade higher. Represented are MF patients with early stage MF (patch and plaque) (N=61).

FIG. 10 shows mortality risk according to TOX mRNA levels in MF (entire patient population). The MF skin biopsies were divided to the TOX high group (TOX level higher than the top sample of the control group) and the TOX low group (level no different than the benign dermatoses). Represented are all MF patients analyzed (N=77).

FIG. 11 shows that TOX mRNA levels in Sezary Cells of Sézary syndrome patients are much higher than in CD4+ T cells from control subjects. TOX mRNA is quantified using real-time quantitative polymerase chain reaction in CD4+ T cells purified from the peripheral blood of patients with Sézary syndrome (N=12, CD4+CD7− T cells), and those with benign dermatoses such as psoriasis (N=7), rosacea (N=5), vitiligo (N=5), and normal skin (N=9). Non-SS: Summary of PSO, ROS, VT and NC samples (N=26).

FIG. 12 shows TOX mRNA levels as a diagnosis marker for Sézary syndrome. TOX mRNA is quantified using real-time quantitative polymerase chain reaction in CD4+ T cells purified from Sézary syndrome (N=12) patients and the benign control CD4+ T cells (N=26).

FIG. 13 shows mortality risk according to TOX mRNA levels in Sézary syndrome. The Sézary syndrome skin biopsies were divided to the TOX high group (TOX level higher than the top sample of the control group) and the TOX low group (level no different than the benign dermatoses). The five year mortality is analyzed using Prism 5 software.

FIG. 14 shows Western blots for TOX protein in cell lines from subjects with T cell malignancy. The level of TOX protein was highly increased in four CTCL cell lines (Hut78; Hut102; HH; SZ4), two T-lineage acute lymphoblastic leukemia cell lines (Jurkat; CCL119), and CD4+ T cells from one patient with Sezary syndrome (SS-5), compared with CD4+ T cells from benign inflammatory skin disorders (Ctr 1, and Ctr 2).

FIG. 15 shows that TOX positive cells are enriched in the CD7− cell populations in peripheral blood from a patient with Sézary syndrome. CD7 is a surrogate negative marker for CTCL. Numbers denote the percentage of cells within the box out of the total population. (A). TOX+ cells represented a higher proportion (6.7%) in PBMC from Sézary syndrome patient, compared with healthy control (1.41%); (B). A marked increase of CD7− cells was observed in PBMC from Sézary syndrome relative to a healthy control. In addition, TOX+ cells were enriched in the CD4+CD7− population. PBMC=Peripheral blood mononuclear cell. (Y-axis=TOX, x-axis=Forward Scatter (FSC)).

### DETAILED DESCRIPTION

The present inventors have identified biomarkers useful for screening for, detecting or diagnosing T cell malignancies. As set out in Example 1, high throughput genomic transcription profiling was used to identify genes differentially expressed in samples from subjects with early mycosis fungoides (eMF) relative to samples from subjects with normal skin or benign skin conditions such as chronic dermatitis. Each of the biomarkers listed in Table 2 was observed to be upregulated in samples from subjects with eMF relative to samples from subjects with chronic dermatitis or normal skin. TOX showed the greatest differential expression between samples from subjects with eMF relative to samples from normal subjects or subjects with chronic dermatitis. The biomarkers listed in Table 2 are therefore useful for screening for, detecting or diagnosing T cell malignancy as well as excluding a diagnosis of T cell malignancy.

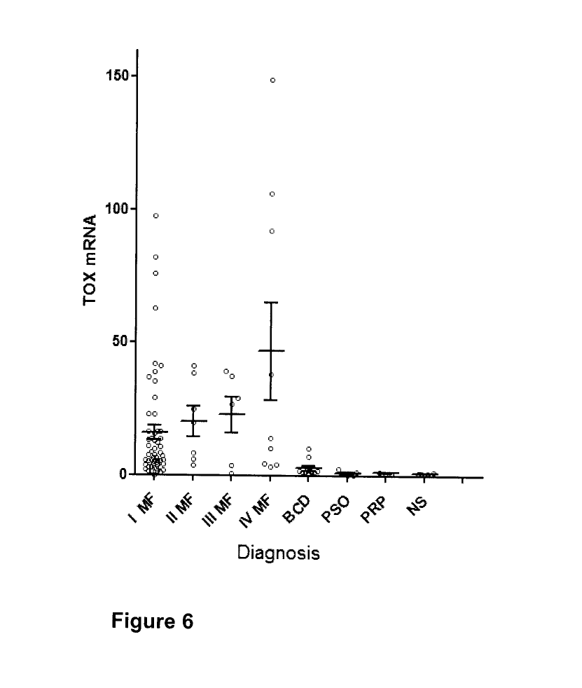

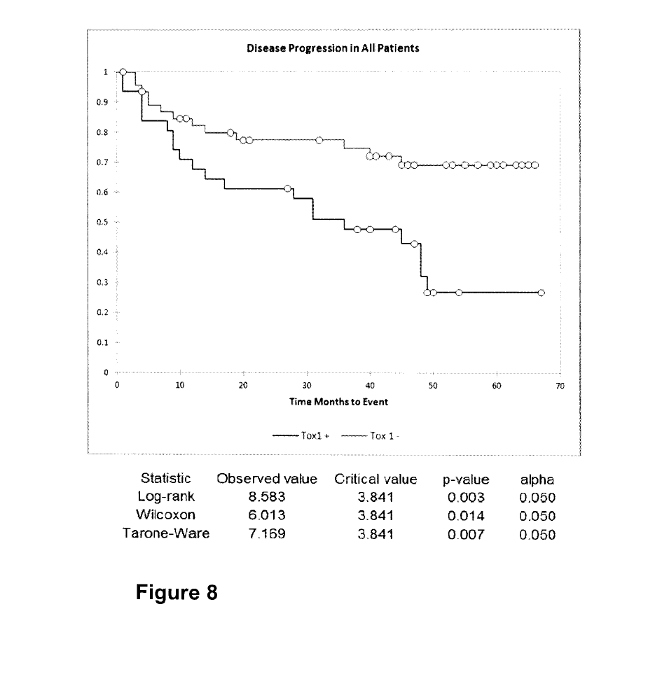

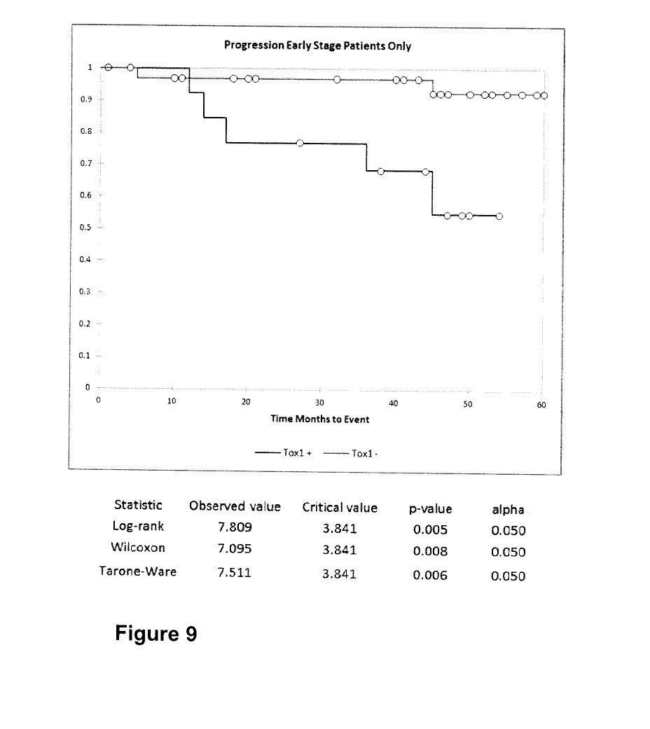

As shown in Example 2, TOX is also useful as a prognostic biomarker for T cell malignancies such as CTCL. More specifically, levels of TOX mRNA were shown to increase with progression of disease from stage I to stage IV (FIG. 6). Receiver Operator Characteristic (ROC) curves presented in FIG. 8 show that binary classification of samples into high and low TOX levels is a statistically significant predictor for the 5-year occurrence of progressing to malignant disease at least 1 numerical grade higher. Remarkably, as shown in FIG. 9 TOX is also a statistically significant predictor of disease severity in early stage disease. Levels of TOX protein were also shown to be elevated cell lines from subjects with T cell malignancies such as CTCL and T-lineage acute lymphoblastic leukemia.

Accordingly, in one aspect the methods described herein are useful for screening for, diagnosing or detecting T cell malignancy in a subject. For example, in one embodiment the method comprises determining a level of TOX in a sample from a subject and comparing the level of TOX in the sample to a control level. In one embodiment an increased level of TOX in the sample relative to the control level indicates that the subject has T cell malignancy.

The methods described herein are also useful for monitoring T cell malignancy in a subject. In one embodiment the methods described herein include determining a level of TOX in a sample from a subject at a first time point and determining a level of TOX in a sample from the subject at a second time point and comparing the level of TOX at the first time point with the level of TOX at the second time point.

The methods described herein are also useful for providing a prognosis for a subject with T cell malignancy. For example, in one embodiment, the methods comprises determining a level of TOX in a sample from the subject and comparing the level of TOX in the sample to a control level wherein a difference or similarity between the level of TOX in the sample and the control level is indicative of the severity of the disease.

### I. Definitions

As used herein, “TOX” refers to the “Thymocyte selection-associated high mobility group box protein” as well as the gene, nucleic acids and/or polypeptides encoding for TOX. In one embodiment, TOX is encoded by the nucleic acid sequences or polypeptide sequences set forth in database identifiers HGNC: 18988; Entrez Gene: 9760; Ensembl: ENSG00000198846 and UniProtKB: O94900. In one embodiment, TOX refers to the gene, nucleic acids and/or polypeptides as generally described in Wilkinson et al. TOX: an HMG box protein implicated in the regulation of thymocyte selection. Nature Immunology 3 (3): 272-80 (2002), hereby incorporated by reference in its entirety. In one embodiment, TOX is a biomarker for T cell malignancy.

The term “biomarker” as used herein refers to a nucleic acid or polypeptide, such as an expression product or fragment thereof, of a gene listed Table 2 which can be used to distinguish subjects with or without T cell malignancy or to provide a prognosis for a subject with T cell malignancy.

As used herein, “T cell malignancy” refers to cancer characterized by the malignant growth of T cells. Examples of T cell malignancy include, but are not limited to, cutaneous T cell lymphoma, peripheral T cell lymphoma and T cell leukemia.

As used herein, “cutaneous T cell lymphoma (CTCL)” refers to cancer characterized by lymphoid malignancies derived from T lymphocytes residing in the skin. Subjects with early stage CTCL may present with a rash or skin irritation, which may eventually form plaques and tumors before metastasizing to other parts of the body as the disease progresses. Malignant cells display mature memory T cell markers (i.e. CD4+CD45RO+) but often lose other mature T cell markers such as CD7 and CD26. Subjects with CTCL typically present with the clinical features described above along with the “atypical” histological characteristics of the CTCL cells. These include a slightly larger or angulated nuclear contour, and migration of these cells into the top layer of the skin, the epidermis. In some cases, the cells of CTCL in the peripheral blood carry a unique, but rare multi-lobulated nuclear shape. However, these morphological changes are often difficult to identify, and over lapping cases often occur with benign inflammatory conditions such as chronic dermatitis or allergic reactions to medications. In some cases, it is possible to diagnose CTCL by testing for rearrangement of the T cell receptor gene. However, T cell clonality sometimes occurs in the benign cases, and often CTCL does not present with T cell clonality.

Examples of CTCL include mycosis fungoides and Sezary syndrome. “Mycosis fungoides (MF)” is the most common form of CTCL. Subjects with MF typically have skin manifestations that resemble common benign skin inflammatory conditions such as psoriasis, chronic dermatitis and may present with rash like patches, tumors, or lesions. Malignancies in MF originate from peripheral memory T cells. Optionally, malignant T cells in subjects with MF exhibit a loss of CD7, CD2, CD3 and/or CD28.

As used herein, “early mycosis fungoides (eMF)” refers to early stage disease characterized by patch and early plaque mycosis fungoides. In one embodiment, eMF refers to stage I disease.

“Sezary syndrome” is a leukemic variant of CTCL with systemic involvement. Subjects with Sezary syndrome typically have abnormally shaped lymphocytes, termed Sezary cells, in the peripheral blood. Malignancies in Sezary syndrome originate from central memory T cells. Cancerous cells in Sezary syndrome are typically much larger than in MF and have cerebriform nucleus, and often have loss of CD7.

T cell malignancies may be staged and/or classified as commonly known in the art. For example, Olsen et al. Blood, 15 Sep. 2007, Vol. 110, No. 6 (incorporated by reference herein in its entirety), describe criteria for the staging and classification of mycosis fungoides and Sezary syndrome. In some embodiments stage I CTCL is characterized by limited plaques, papules, or eczematous patches covering less than 10% of the skin surface and no clinically abnormal peripheral lymph nodes or malignancies in visceral organs. In some embodiments, stage II CTCL is characterized by the generalized plaques, papules, or erythematous patches covering greater than 10% or more of the skin surface. In some embodiments, stage III CTCL is characterized by development of tumors, whereas stage IV CTCL refers to the involvement of blood, that is, the CTCL cells have become circulating, becoming leukemic in nature.

The term “sample” as used herein means any sample containing T cells including, but not limited to, biological fluids, tissue extracts, freshly harvested cells, and lysates of cells which have been incubated in cell cultures for which the presence or absence of one or more biomarkers is determined. In one embodiment, the sample is a tissue sample or blood sample. In one embodiment, the tissue sample is a skin sample, such as a biopsy of a skin lesion. In one embodiment, the sample comprises peripheral blood mononuclear cells (PBMCs). In one embodiment, the sample comprises CD4+ T cells. In one embodiment, the sample is from an individual subject. Alternatively, the sample may be a pooled sample from a plurality of subjects. As used herein, the term “sample” includes biological samples, or fractions thereof, that have been processed or treated such as to remove, inactivate or isolate constituents in the sample. In certain embodiments, the samples are processed prior to detecting the biomarker level. For example, a sample may be fractionated (e.g. by centrifugation or using a column for size exclusion), concentrated or proteolytically processed such as trypsinized, depending on the method of determining the level of biomarker employed.

The term “subject” as used herein refers to any member of the animal kingdom, preferably a human being, including a subject that has, or is suspected of having, a T cell malignancy.

The phrase “screening for, diagnosing or detecting T cell malignancy” refers to a method or process that aids in the determination of whether a subject has or does not have T cell malignancy that involves determining the level of one or more of the biomarkers listed in Table 2. For example, in one embodiment detection of increased levels of TOX in a sample from a subject relative to a control level indicates that the subject has T cell malignancy. In one embodiment, detection of increased levels of TOX and increased levels of one or more additional biomarkers from Table 2 relative to a control level is indicative that the subject has T cell malignancy.

As used herein, “providing a prognosis” refers to a method or process that aids in predicting the clinical outcome or likely progression of disease caused by T cell malignancy in a subject that involves determining the level of one or more of the biomarkers listed in Table 2. Examples of providing a prognosis include, but are not limited to, estimating mortality or survival within a particular time-span or progression of T cell malignancy in a subject to a more severe form of the disease, such as progressing to stage II, stage III or stage IV disease. For example, in one embodiment the magnitude of the level of TOX in a sample from a subject compared to a control level is indicative of the severity of the disease. In some embodiments, “providing a prognosis” includes predicting the progression or remission of T cell malignant disease.

As used herein, the term “monitoring T cell malignancy” refers to a method or process that aids in the determination of any change in the status or severity of disease caused by T cell malignancy in a subject that involves detecting one or more of the biomarkers listed in Table 2. In some embodiments, the methods involve comparing the level of one or more biomarkers in a sample taken from a subject at a first time point with the level of one or more biomarkers in a sample taken form a subject at a later time point. In one embodiment, detecting an increase in the level of TOX in a sample from the subject is indicative of an increase in the severity of disease in the subject. In one embodiment, detecting a decrease in the level of TOX in a sample from the subject is indicative of a decrease in the severity of the disease. For example, in one embodiment the methods described herein are useful for determining whether a subject is responsive to treatment with one or more chemotherapeutic agents. In one embodiment, an increase in the level of TOX in a sample from a subject post-treatment compared to a control level (such as a level of TOX in a sample from the subject prior to treatment) is indicative that the subject is not responding or is responding poorly to treatment. In one embodiment, a decrease in the level of TOX in a post treatment sample compared to a control level (such as a level of TOX in a sample from the subject prior to treatment) is indicative that the subject is responding to treatment.

The term “level” as used herein refers to an amount (e.g. relative amount or concentration) of biomarker that is detectable or measurable in a sample. For example, the level can be a copy number, concentration such as μg/L or a relative amount such as 1.0, 1.5, 2.0, 2.5, 3, 5, 10, 15, 20, 25, 30, 40, 60, 80 or 100 times a control level. Optionally, the term level includes the level of a biomarker normalized to an internal normalization control, such as the expression of a housekeeping gene. In one embodiment, the housekeeping gene is beta actin. In one embodiment, the level of a biomarker is normalized to nucleic acid or a polypeptide that is present in the sample type being assayed, for example a house keeping gene protein, such as beta-actin, glyceraldehyde-3-phosphate dehydrogenase, or beta-tubulin, or total protein, e.g. any level which is relatively constant between subjects for a given volume.

The term “control level” refers to the level of a biomarker that is representative of a sample or group of samples from a subject or group of subjects for whom the status with respect to T cell malignancy is known. In one embodiment, the control level refers to the level of a biomarker that is representative of a sample or group of samples from a subject or group of subjects without T cell malignancy, optionally without CTCL. In one embodiment, the control level refers to a cut-off value, wherein subjects with a biomarker level at or below such a value are likely not to have T cell malignancy, and subjects with a biomarker level above such a value have or are likely to have T cell malignancy. In another example, the control can be a value that corresponds to the median level of the biomarker in a set of samples from subjects without T cell malignancy. In one embodiment the control level is an average or median level in a sample or group of samples from a subject or group of subjects. In some embodiments, the control level is representative of the level of biomarker in subjects with a particular stage of disease, such as stage I, stage II, stage III or stage IV T cell malignancy. In one embodiment, the control level is a predetermined or standardized control level. In one embodiment, the level of TOX in the sample that is indicative of T cell malignancy is at least 1.5, 2.0, 2.5, 3.0, 3.0, 3.5, 4.0, 4.5, 5.0, 6.0, 7.0, 8.0, 9.0, 10, 15, 20 or 25 times greater than the control level.

The term “antibody” as used herein is intended to include monoclonal antibodies, polyclonal antibodies, and chimeric antibodies, and fragments thereof that retain binding activity. The antibody may be from recombinant sources and/or produced in transgenic animals. Antibodies can be fragmented using conventional techniques. For example, F(ab′)2 fragments can be generated by treating the antibody with pepsin. The resulting F(ab′)2 fragment can be treated to reduce disulfide bridges to produce Fab′ fragments. Papain digestion can lead to the formation of Fab fragments. Fab, Fab′ and F(ab′)2, scFv, dsFv, ds-scFv, dimers, minibodies, diabodies, bispecific antibody fragments and other fragments can also be synthesized by recombinant techniques.

The term “detection agent” as used herein refers to any molecule or compound that binds to a biomarker as described herein, including polypeptides such as antibodies, nucleic acids and peptide mimetics. The “detection agent” can for example be coupled to or labeled with a detectable marker. The label is preferably capable of producing, either directly or indirectly, a detectable signal. For example, the label may be radio-opaque or a radioisotope, such as ³H, ¹⁴C, ³²P, ³⁵S, ¹²³I, ¹²⁵I, ¹³¹I; a fluorescent (fluorophore) or chemiluminescent (chromophore) compound, such as fluorescein isothiocyanate, rhodamine or luciferin; an enzyme, such as alkaline phosphatase, beta-galactosidase or horseradish peroxidase; an imaging agent; or a metal ion. Examples of detection agents useful for the methods described herein include antibodies that selectively bind the TOX protein and nucleic acid primers or probes that selectively bind nucleic acid molecules that code for the TOX protein.

### II. Diagnostic and Prognostic Methods for T Cell Malignancies

In one aspect, there is provided a method of screening for, diagnosing or detecting T cell malignancy in a subject. In one embodiment, the method comprises:
(a) determining a level of TOX in a sample from the subject; and
(b) comparing the level of TOX in the sample to a control level, wherein an increased level of TOX in the sample relative to the control level indicates that the subject has T cell malignancy.

In another aspect, there is provided a method of monitoring T cell malignancy in a subject comprising:
(a) determining a level of TOX in a sample from the subject at a first time point;
(b) determining a level of TOX in a sample from the subject at a second time point and comparing the level of TOX in the sample at the first time point with the level of TOX in the sample at the second time point.

In another aspect there is provided a method of providing a prognosis for a subject with T cell malignancy comprising:
(a) determining a level of TOX in a sample from the subject; and
(b) comparing the level of TOX in the sample to a control level.

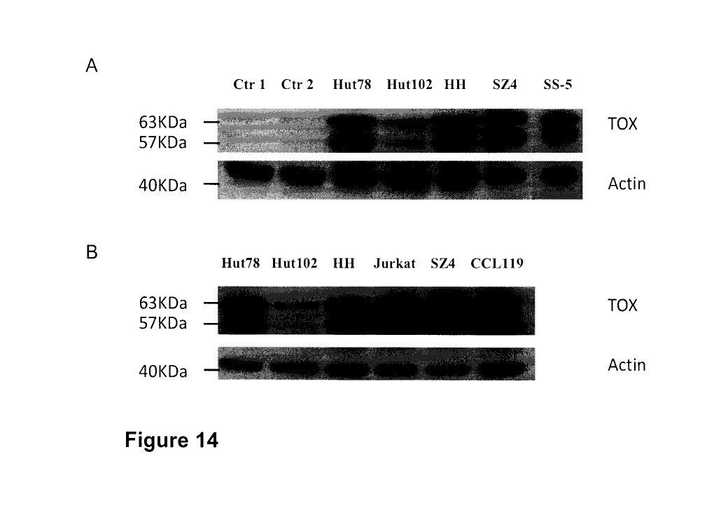

In some embodiments of the methods described herein, the T cell malignancy is cutaneous T cell Lymphoma (CTCL), peripheral T cell lymphoma or T cell leukemia. As shown in Example 1, TOX has been identified as a biomarker for T cell malignancy such as mycosis fungoides and Sezary syndrome. As shown in FIG. 14, TOX has also been shown to be overexpressed relative to controls in other T cell malignancies such as acute lymphoblastic leukemia.

Some embodiments of the methods described herein involve determining the level of one or more biomarkers in a sample from a subject. Optionally, the methods described herein further comprise obtaining a sample from the subject. In a preferred embodiment, the sample comprises one or more T cells from a subject, such as CD4+ T cells. In one embodiment the sample is a tissue sample. In some embodiments, the sample is a skin sample or a blood sample. Tissue samples may be obtained from a subject using biopsy techniques known in the art such as by using a punch biopsy or needle biopsy. Preferably, tissue samples are obtained from areas of the subject thought to harbor malignant T cells, such as areas of skin exhibiting manifestations of the disease such as dermatitis or inflammation. In one embodiment, the sample comprises peripheral blood mononuclear cells (PBMCs). In some embodiments, the sample is frozen or processed to remove cell debris or material that may interfere with testing the sample for the expression of biomarkers. For example, in some embodiments a blood sample is centrifuged to separate the sample into plasma and blood cells. In some embodiments, a tissue sample is processed to dissociate the tissue into individual cells or to isolate cellular components such as proteins or nucleic acids.

The level of the biomarkers described herein such as TOX may be determined in the sample using a variety of methods known to a person of skill in the art. For example, in some embodiment the methods described herein include testing the sample for the expression of TOX. In some embodiment testing the sample for the expression of TOX comprises contacting the sample with a detecting agent. In some embodiments, determining the level of TOX in the sample involves testing the sample for a nucleic acid encoding for all or part of the TOX protein. In some embodiments, determining the level of TOX in the sample involves testing the sample for all or part of the TOX protein.

Preferred embodiments for determining the level of biomarkers such as TOX in a sample according to the methods described herein include immunohistochemistry, immunofluorescence and/or flow cytometry based methods that use antibodies that selectively bind to a biomarker protein, or fragment thereof. Other preferred embodiments for determining the level of a biomarker such as TOX in a sample include detecting the biomarker at the transcriptional (mRNA) level such as by using nucleic acid primers or probes that hybridize to sequences encoding all of part of the biomarker. In some embodiment, the methods described herein include the use of RT-PCR, microarrays, ARMS-based PCR, RNase protection assays, Taqman assays and the like. Optionally, the level of TOX and/or one or more additional biomarkers associated with T cell malignancies selected from Table 2 may be determined using the methods described herein.

In one embodiment, the methods of the invention involve the detection of nucleic acid molecules encoding a biomarker such as TOX. Those skilled in the art can construct nucleotide probes for use in the detection of nucleic acid sequences encoding biomarkers in samples. Suitable probes include nucleic acid molecules based on nucleic acid sequences encoding at least 5 sequential amino acids from regions of the biomarker, preferably 15 to 30 nucleotides. In one embodiment, the probes are useful for detecting nucleic acid molecules encoding for a biomarker in a microarray. A nucleotide probe may be labeled with a detectable substance such as a radioactive label which provides for an adequate signal and has sufficient half-life such as ³²P, ³H, ¹⁴C or the like. Other detectable substances which may be used include antigens that are recognized by a specific labeled antibody, fluorescent compounds, enzymes, antibodies specific for a labeled antigen, and luminescent compounds. An appropriate label may be selected having regard to the rate of hybridization and binding of the probe to the nucleotide to be detected and the amount of nucleotide available for hybridization. Labeled probes may be hybridized to nucleic acids on solid supports such as nitrocellulose filters or nylon membranes as generally described in Sambrook et al, 1989, Molecular Cloning, A Laboratory Manual (2nd ed.). The nucleic acid probes may be used to detect genes, preferably in human cells, that encode for a biomarker. In one embodiment, the nucleic acid probes are used for the screening, diagnosis, prognosis or monitoring of T cell malignancies in a subject.

The probe may be used in hybridization techniques to detect genes that encode biomarker proteins such as TOX protein. The technique generally involves contacting and incubating nucleic acids obtained or derived from a sample from a subject with a probe under conditions favorable for the specific annealing of the probes to complementary sequences in the nucleic acids. After incubation, the non-annealed nucleic acids are removed, and the presence of nucleic acids that have hybridized to the probe if any are detected.

The detection of nucleic acid molecules may involve the amplification of specific gene sequences using an amplification method such as polymerase chain reaction (PCR), followed by the analysis of the amplified molecules using techniques known to those skilled in the art. Suitable primers can be routinely designed by one of skill in the art.

Hybridization and amplification techniques described herein may be used to assay qualitative and quantitative aspects of expression of a gene encoding a biomarker such as TOX. For example, RNA may be isolated from a cell type or tissue such as a tissue sample or blood sample, and tested utilizing the hybridization (e.g. standard Northern analyses) or PCR techniques such as RT-PCR or real time RT-PCR. The techniques may be used to detect differences in transcript size which may be due to normal or abnormal alternative splicing. Optionally the techniques described herein include reverse-transcribing mRNA into cDNA and detecting one or more cDNAs encoding for a biomarkers listed in Table 2.

In some embodiment, the primers and probes may be used in the above described methods in situ i.e. directly on tissue sections (fixed and/or frozen) of patient tissue obtained from biopsies or resections.

In some embodiments the methods described herein optionally include extracting nucleic acid molecules comprising a biomarker gene or portion thereof from a sample from the subject. In some embodiment, the methods include amplifying the extracted nucleic acid molecules using the polymerase chain reaction, optionally RT-PCR.

In another aspect, the methods described herein involve the detection of a protein biomarker. In one embodiment, the protein biomarker is detected using a detection agent such as an antibody that selectively binds to the protein. In one embodiment, the protein biomarker is detected using protein mass spectrometry such as LC-MS, optionally quantitative protein mass spectrometry. In one embodiment, the protein biomarker is the TOX protein.

Antibodies to biomarkers such as TOX may be prepared using techniques known in the art. For example, by using a peptide of the biomarker protein, polyclonal antisera or monoclonal antibodies can be made using standard methods. A mammal, (e.g., a mouse, hamster, or rabbit) can be immunized with an immunogenic form of the peptide which elicits an antibody response in the mammal. Techniques for conferring immunogenicity on a peptide include conjugation to carriers or other techniques well known in the art. For example, the protein or peptide can be administered in the presence of adjuvant. The progress of immunization can be monitored by detection of antibody titers in plasma or serum. Standard ELISA or other immunoassay procedures can be used with the immunogen as antigen to assess the levels of antibodies. Following immunization, antisera can be obtained and, if desired, polyclonal antibodies isolated from the sera.

To produce monoclonal antibodies, antibody producing cells (lymphocytes) can be harvested from an immunized animal and fused with myeloma cells by standard somatic cell fusion procedures thus immortalizing these cells and yielding hybridoma cells. Such techniques are well known in the art, (e.g., the hybridoma technique originally developed by Kohler and Milstein (Nature 256, 495-497 (1975)) as well as other techniques such as the human B-cell hybridoma technique (Kozbor et al., Immunol. Today 4, 72 (1983)), the EBV-hybridoma technique to produce human monoclonal antibodies (Cole et al. Monoclonal Antibodies in Cancer Therapy (1985) Allen R. Bliss, Inc., pages 77-96), and screening of combinatorial antibody libraries (Huse et al., Science 246, 1275 (1989)). Hybridoma cells can be screened immunochemically for production of antibodies specifically reactive with the peptide and the monoclonal antibodies can be isolated.

Antibodies that are selective for the biomarkers described herein, or derivatives, such as enzyme conjugates or labeled derivatives, may be used to detect biomarkers in various samples (e.g. biological materials). They may be used as diagnostic or prognostic reagents and they may be used to detect abnormalities in the level of protein expression, or abnormalities in the structure, and/or temporal, tissue, cellular, or subcellular location of the biomarker. In vitro immunoassays may also be used to assess or monitor the efficacy of particular therapies. The antibodies of the invention may also be used in vitro to determine the level of expression of a gene encoding the biomarker in cells genetically engineered to produce the biomarker protein.

The antibodies may be used in any known immunoassays which rely on the binding interaction between an antigenic determinant and the antibodies. Examples of such assays are radioimmunoassays, enzyme immunoassays (e.g. ELISA), immunofluorescence, immunoprecipitation, latex agglutination, hemagglutination, and histochemical tests. The antibodies may be used to detect and quantify the biomarker in a sample in order to determine its role in T cell malignancy and/or to diagnose T cell malignancy or provide a prognosis for a subject with T cell malignancy. Optionally the antibodies are used in combination with techniques such as Fluorescence Activated Cell Sorting (FACS) in order to determine the level of expression of a biomarker.

Cytochemical techniques known in the art for localizing antigens using light and electron microscopy may be used to detect protein biomarkers such TOX. Generally, an antibody of the invention may be labeled with a detectable substance and the protein may be localised in tissues and cells based upon the presence of the detectable substance. Examples of detectable substances include, but are not limited to, the following: radioisotopes (e.g., ³H, ¹⁴C, ³⁵S, ¹²⁵I, ¹³¹I), fluorescent labels (e.g., FITC, rhodamine, lanthanide phosphors), luminescent labels such as luminol; enzymatic labels (e.g., horseradish peroxidase, beta-galactosidase, luciferase, alkaline phosphatase, acetylcholinesterase), biotinyl groups (which can be detected by marked avidin e.g., streptavidin containing a fluorescent marker or enzymatic activity that can be detected by optical or calorimetric methods), predetermined polypeptide epitopes recognized by a secondary reporter (e.g., leucine zipper pair sequences, binding sites for secondary antibodies, metal binding domains, epitope tags). In some embodiments, labels are attached via spacer arms of various lengths to reduce potential steric hindrance. Antibodies may also be coupled to electron dense substances, such as ferritin or colloidal gold, which are readily visualised by electron microscopy.

The antibody or sample may be immobilized on a carrier or solid support which is capable of immobilizing cells, antibodies etc. For example, the carrier or support may be nitrocellulose, or glass, polyacrylamides, gabbros, and magnetite. The support material may have any possible configuration including spherical (e.g. bead), cylindrical (e.g. inside surface of a test tube or well, or the external surface of a rod), or flat (e.g. sheet, test strip). Indirect methods may also be employed in which the primary antigen-antibody reaction is amplified by the introduction of a second antibody, having specificity for the antibody reactive against the biomarker protein.

Some embodiments of the methods described herein involve comparing the level of a biomarker in a sample to a control level. A skilled person will appreciate selecting a suitable control level in order to diagnose or provide a prognosis for a subject with T cell malignancy. The control level will also depend on the desired specificity and sensitivity of the diagnosis or diagnosis.

In some embodiments described herein the methods comprise screening for, diagnosing, detecting or monitoring T cell malignancy in a subject and then treating a subject identified as having a T cell malignancy for the disease. In one embodiment, the methods described herein include making a treatment decision based on the level of TOX in a sample from the subject. For example, in one embodiment, the methods described herein include treating a subject identified as having a T cell malignancy with one or more anticancer therapies and/or antineoplastic agents. In one embodiment, the methods described herein further comprise administering to a subject identified as having a T cell malignancy subject one or more chemotherapeutic and/or antineoplastic agents. Examples of chemotherapeutic and/or antineoplastic agents include, but are not limited to, alkylating agents such as topical nitrogen mustard (e.g. chlorambucil), histone deacetylase (HDAC) inhibitors such as Vorinostat, suberoylanilide hydroxamic acid (SAHA), and Romidepsin as well as other antineoplastic agents such as Denileukin diftitox or Bexarotene. In one embodiment the methods described herein include administering to a subject identified as having a T cell malignancy subject one or more anticancer therapies suitable for treating T cell malignancy such as, but not limited to, long-wave ultraviolet B therapy, total body or local radiation therapy or retinoic acid.

In some embodiments, the methods described herein are useful for monitoring a subject with T cell malignancy. In one embodiment, an increase in the level of TOX is indicative of an increase in severity of disease and a decrease in the level of TOX is indicative of a decrease in severity of disease. For example, in one embodiment the method involves comparing the levels of a biomarker in samples taken from a subject at different time points. In one embodiment, the method comprises determining a level of TOX in a sample from the subject at a first time point and determining a level of TOX in a sample from the subject at a second time point and comparing the level of TOX in the sample at the first time point with the level of TOX in the sample at the second time point. In one embodiment, an increase in the level of TOX is indicative of the presence of T cell malignancy or of an increase in severity of disease. In one embodiment, a decrease in the level of TOX is indicative of a decrease in severity of disease. In some embodiments, the magnitude of the increase or decrease in the level of TOX is indicative of the magnitude of the increase or decrease in the severity of the disease. Optionally, the subject is undergoing treatment for T cell malignancy and the method is used to monitor a response of the subject to the treatment.

In some embodiments, the methods described herein are useful for providing a prognosis for a subject with T cell malignancy that involve comparing the level of a biomarker such as TOX in a sample from a subject to a control level. In one embodiment, the control level is a level that is representative of the level of a biomarker in a control subject or population of control subjects. In one embodiment the control level is representative of the level of a biomarker in a population of control subjects with a particular outcome such as mortality rates or a particular disease state, such as cancer stage.

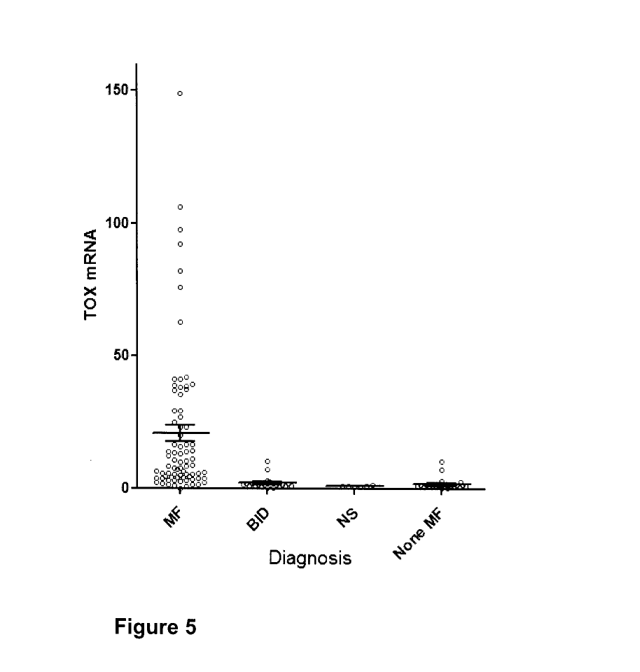

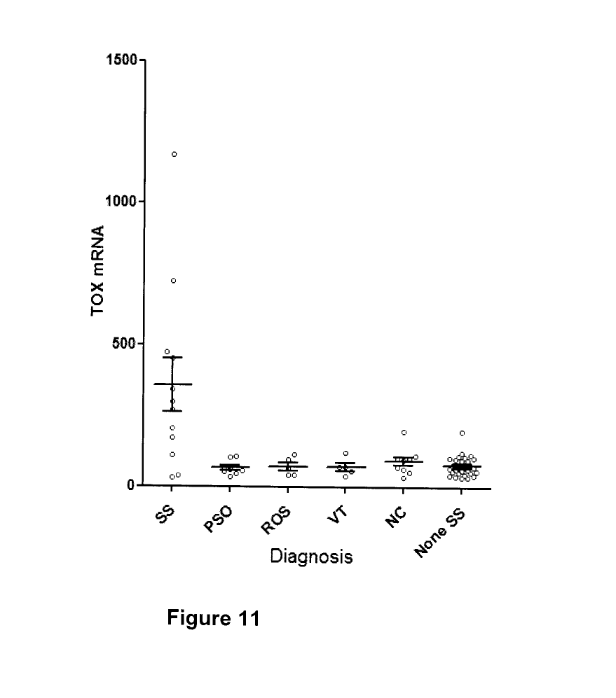

For example, the control can be a predetermined cut-off level or threshold wherein subjects with a level of biomarker greater than the cut-off level are identified as having T cell malignancy. As shown in FIGS. 5 and 11, subjects with MF or Sezary syndrome have higher TOX mRNA levels relative to control samples and as shown in FIG. 14, subjects with T cell malignancy have higher TOX protein levels relative to control samples. Selecting a value for a control level, such as a cut-off value, wherein subjects having an increased level of one of more biomarkers disclosed herein is useful for identifying subjects as having T cell malignancies or for providing a prognosis for the disease. As shown in FIG. 6, levels of the biomarker TOX increase with the progression of mycosis fungoides disease from stage I to stage IV. Accordingly, in one embodiment the control level is representative of a level of TOX in one or more samples from subjects with stage I, stage II, stage III or stage IV T cell malignancy, such as the average or median level of TOX in a population of subjects with stage I, stage II, stage III or stage IV T cell malignancy.

A skilled person will appreciate that when comparing the levels of a biomarker in a sample to a control level, the diagnosis or prognosis will depend on the severity of disease in the population of subjects that are selected to form a control group. In one embodiment, subjects with an increased level of TOX relative to the control group have a worse prognosis with respect to the severity of the disease relative to the control group. In one embodiment, subjects with a decreased level of TOX relative to the control group have a better prognosis with respect to the severity of the disease relative to the control group. In some embodiments, the prognosis is the likelihood of the subject progressing to a least one numerical grade higher of T cell lymphoma. In some embodiments, the prognosis is the likelihood of mortality from the disease, such as mortality within a 5-year time frame.

### III. Kits

In one aspect, there is provided a kit useful for conducting a method as described herein, such as for diagnosing, monitoring or providing a prognosis for T cell malignancies. In one embodiment, the kit includes one or more reagents suitable for conducting a method as described herein. Optionally, the kit may include instructions for use and/or containers suitable for the storing the reagents.

In one embodiment, the kit includes a detection agent suitable for detecting a biomarker listed in Table 2. In one embodiment, the kit includes a detection agent suitable for detecting TOX. In one embodiment, the kit includes a detection agent specific for TOX and at least one additional detection agent specific for a biomarker listed in Table 2. In one embodiment, the kit includes 2, 3, 4, 5, or more than 5 detection agents suitable for detecting 2, 3, 4, 5, or more than 5 biomarkers listed in Table 2. Optionally, the kits also include one or more detection agents for detecting CD7, CD2, CD3 and/or CD28. In one embodiment, the kit comprises buffers or enzymes useful for practicing the methods described herein. In one embodiment, the kit comprises control samples with known level of TOX.

The following non-limiting examples are illustrative of the present disclosure:

### EXAMPLES

### Example 1

### Identification of Biomarkers for T Cell Malignancy

### Materials and Methods

Skin Biopsies of eMF, BID and NS

Lesional skin biopsies were obtained using 3 mm punches under local anesthesia from 21 patients with eMF (patch and early plaque (Olsen, Vonderheid et al. 2007) recruited from the Skin Lymphoma Clinic of British Columbia Cancer Agency and the outpatient dermatology clinics of the Vancouver General Hospital (N=12) in the Department of Dermatology and Venerology, First Affiliated Hospital, Peking University, Beijing, China (N=9), with approval by the Clinical Ethics Board of both institutions, in accordance with the Declaration of Helsinki principles (Molecular Disease Markers, Approval Number C98-0493). Patients were diagnosed and staged based on clinical history, physical examination, histology findings and immunophenotypic characteristics according to previously described criteria (Olsen, Vonderheid et al. 2007). Patients were enrolled with stage IA-IB disease. Patients were 28-82 years of age old with an average age at 49.5. Patient demographics are listed in Table 1. As controls, skin biopsies were obtained from healthy volunteers (N=21) and 15 subjects with benign inflammatory dermatoses (BID), including psoriasis (n=6), chronic dermatitis (n=6) and pityriasis rubra pilaris (n=3). The biopsies were placed into RNALater solutions ((Invitrogen, Burlington, ON, Canada) and stored at −20° C. until RNA extraction.

### RNA Isolation and Gene Transcription Profiling

Total cellular RNA was extracted using the RNeasy Mini Kit (Qiagen Inc., Mississauga, ON, Canada) according to the manufacturer's instructions. For preparing fluorescently labeled probes for DNA microarray experiments, 500 ng RNA were reverse-transcribed and linearly amplified by in vitro transcription in the presence of fluorescent-labeled CTP using the Low RNA Input Linear Amplification Kit, following the manufacturer's instructions (Agilent Technologies, Canada). Two color transcriptome experiments were performed, with each experimental sample (5 eMF, 5 CD, and 15 NS) labeled with Cy5. As a reference, a common Cy3-labeled reference sample was prepared from a mixture of healthy skin samples by mixing equal proportions of the 15 skins biopsies, and used for every experimental sample (N=25). The Whole Human Genome Oligo microarrays (G4112F, Agilent Technologies, Canada) comprising 41,059 60-nt oligonucleotide probes, were used for the hybridization. The Agilent DNA Microarray Scanner was used for image acquisition and initial intensity analyses for Cy5 and Cy3 signals from each probe, separately. After quartile normalization, the samples were analyzed using GeneSpring software version 7.3. Microarrays that passed the standard for quality control purposes were used for subsequent analysis.

### Clustering and Pathway Analysis

Two different algorithms were adopted to evaluate contribution of gene pathways to the transcriptional differentiation of samples. 1) GO analysis. Gene Ontology (GO) is a collaborative and comprehensive gene annotation resource compiled by the Gene Ontology Consortium (Ashburner, Ball et al. 2000). GO annotations were obtained from Agilent microarray platform and the enrichment of biological annotation terms in selected gene lists were statistically analyzed with Database for Annotation, Visualization and Integrated Discovery (DAVID) Bioinformatics Resources 6.7 (Huang da, Sherman et al. 2009a; Huang da, Sherman et al. 2009b). The enriched annotation terms associated with the selected gene list gives insights about the biological themes behind the transcriptional profiles. After the enriched GO term lists have been generated, a modular enrichment analysis (MEA) tool was used to classify these lists and to avoid the highly redundant annotations. All annotation sets were ranked by enrichment score and Benjamini adjusted P value. 2) Molecular pathway analysis. Selected gene lists were mapped to Biocarta (Biocarta, San Diego, Calif.), with 274 molecular pathways involved in adhesion, apoptosis, cell activation, cell-cycle regulation, cell signaling, cytokines and chemokines, developmental biology, hematopoiesis, immunology, metabolism, and neurosciences. The enrichment of pathways was also analyzed using DAVID Bioinformatics Resources 6.7.

### Identification of Differentially Expressed Genes in eMF

Given the large number of differentially expressed genes the 41K transcripts expression profile study would generate, a robust data analysis was performed with the following strategy. First, only the genes with expression intensities greater than 100 in at least 5 of the 25 samples tested are analyzed further to avoid false positives from low-abundance genes. Second, we applied stringent filtering methods using Bonferroni correction of p values set at 0.05, and fold changes set at >2. Finally, additional filtering was performed by removing all genes that showed significant (2 fold or more) over expression in benign inflammatory dermatoses (such as chronic dermatitis), leaving only 19 genes showing selective enrichment in eMF but not in CD when compared with NS.

Confirmation with Quantitative Real-Time Polymerase Chain Reaction

RNA was reverse transcribed using random primers and SuperScript III reverse transcriptase (Invitrogen, Burlington, ON, Canada). Real-time polymerase chain reaction (PCR) was performed and analyzed, with GAPDH and 18S genes as the internal controls.

The results are expressed as copies of the specific genes per 1000 copies of GAPDH. The formula the calculation of transcript abundance was as previous reported ((Su, Dorocicz et al. 2003; Wang, Su et al. 2011).

### Immunofluorescence and Immunohistochemistry Studies

Cryosections of lesional skin from eMF patients and controls were fixed with 4% ice-cold paraformaldehyde. After permeabilization and blocking, slides were incubated with rabbit anti-TOX polycloncal antibody (Sigma-Aldrich, CA, USA) and mouse monoclonal anti-human CD4 antibody (Dako Inc., Mississauga, ON, Canada). This was followed by double staining with Alexa-594 conjugated secondary antibody (Red) and Alexa-488 conjugated secondary antibody (Green) (Invitrogen, CA, USA). Cell nuclei were counterstained with DAPI before being mounted with Fluorescence Mounting Medium (DAKO, ON, Canada). Images were collected and processed by fluorescence microscope. Immunohistochemistry was performed using methods previously reported (Dai, Makretsov et al. 2003; Zhou, Dai et al. 2005; Tang, Dai et al. 2006; Tang, Su et al. 2008; Wang, Jiang et al. 2010).

### Statistical Analysis

GeneSpring (version 7.3) was used for transcriptome analysis, including the filtering based after Bonferroni correction for multiple testing, clustering analysis, pathway analysis as well as heat-map construction.

### Results

### Gene Expression Profiles of Early Stage MF

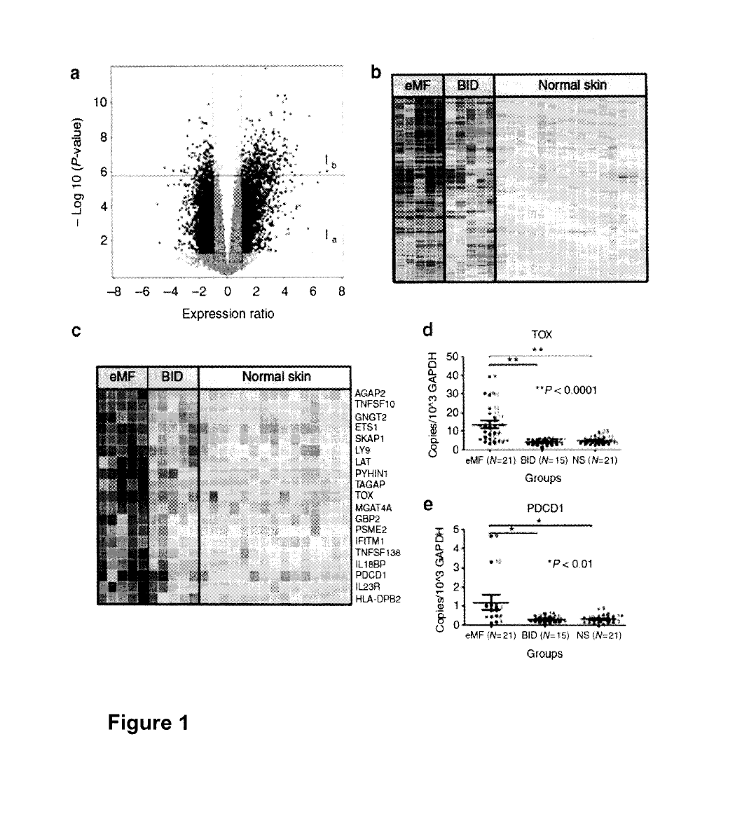

In order to determine the gene expression characteristics of eMF, two-colored comparative transcriptome analysis was performed on 5 eMF samples (Table 1), 5 chronic dermatitis (CD) samples, and 15 normal skin (NS) samples, using a common internal control that was prepared by mixing equal proportions of the 15 NS messenger RNA samples. After verifying the qualities of the 25 microarrays were adequate, a two-stepped data-mining approach was employed using GeneSpring bioinformatic software (version 7.3). First, a volcano plot was performed to select the transcripts that show significant differential expression in eMF compared with NS (>2 fold, p<0.05 after Bonferroni correction for multi-testing, FIG. 1a), yielding 486 transcripts (representing 349 unique genes, Table 4. Almost all of the differentially expressed genes in MF skin biopsies previously reported by others (Shin, Monti et al. 2007; Litvinov, Jones et al. 2010) are confirmed in this list. The highest expressed genes, such as CXCL10, CXCL9, GZMB, KIR2DS2, and IFNG, are involved in chronic inflammation and immune activation, suggesting presence of broad-spectrum immune activation in the eMF lesions. This was supported by examining the pathways enriched in eMF (Table 5), using Database for Annotation, Visualization and Integrated Discovery (DAVID) Bioinformatics Resources 6.7 (Huang da, Sherman et al. 2009a; Huang da, Sherman et al. 2009b). The eMF-overexpressed genes point to significantly enriched pathways of immune response against target cells, Lck and Fyn tyrosine kinase pathway, antigen processing and presentation, caspase cascade in apoptosis, as well as Th1/Th2 differentiation.

Differentially Upregulated Genes in eMF Compared with CD

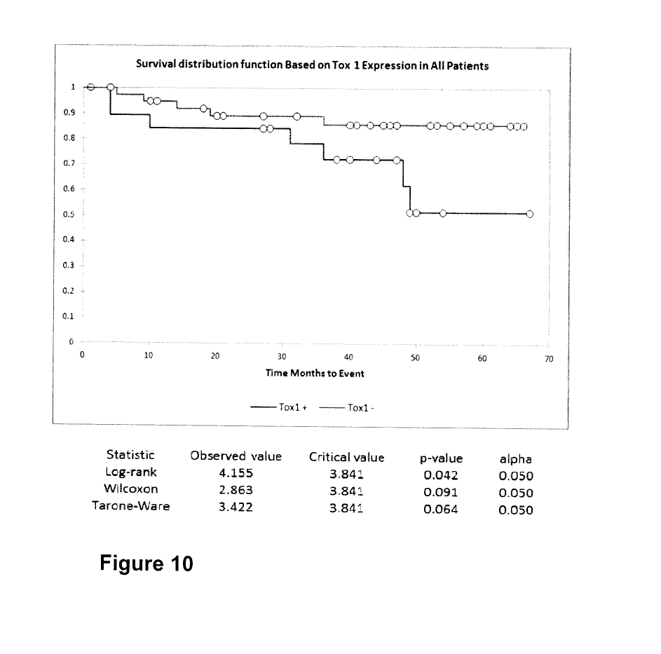

Further examination of the eMF enriched genes showed that the vast majority of them (N=330) were not specific for eMF, since they also showed significant (albeit at different degrees) up-regulation in chronic dermatitis (FIG. 1B, Table 4). To select the more likely candidates of eMF selective genes, a second step of filtering was performed by filtering out these genes, leaving 19 genes with greater than 2 fold up-regulation (p<0.05 t test after Bonferroni correction for multiple testing) in eMF but with no significant up-regulation in CD when compared with NS (Table 2, FIG. 10).

One of these genes, TOX, is a critical regulator of early T cell development, specifically during the transition from CD4+CD8+ precursors to CD4+ T cells. However, upon completion of this process, it is tightly suppressed, so that normal mature CD4+ cells do not have significant expression of this protein (Wilkinson, Chen et al. 2002). Of the remaining genes, 9 genes (IL23R, TAGAO, HLADPB2, LY9, IL18BP, TNFSF13B, IFITM1, TNFSF10, and LAT) are involved in immune regulation; whereas 7 genes are involved in cell signal transduction (PYHIN1, SKAP1, GBP2, ETS1, AGAP2, GNGT2, and PSME2). One gene, MGAT4A, regulates cell adhesion. One gene, PDCD1, is a pro-apoptosis regulator.

To verify the findings, the two most significantly unregulated eMF gene markers, TOX and PDCD1 were analyzed by RT-PCR using an expanded sample set that included 21 eMF samples (Table 1), 15 BID (including 6 CD, 6 psoriasis and 3 pityriasis rubra pilaris), and 21 NS samples. As shown in FIGS. 1D and 1E, both genes, especially TOX, demonstrated highly significant up-regulation compared with both BID and NS. Further, receiver operating characteristic (ROC) curves were used to estimate the specificity and sensitivity of these gene transcripts to distinguish eMF from their benign mimickers. When the specificity was set at 100%, the sensitivity was 71.3% for TOX and 60% for PDCD1. In general, TOX and PDCD1 showed positive correlation with each other although two samples showed increased TOX expression but not PDCD1 expression (Table 1).

### TOX Specifically Labels CD4 T Cells in eMF but not in CD or NS

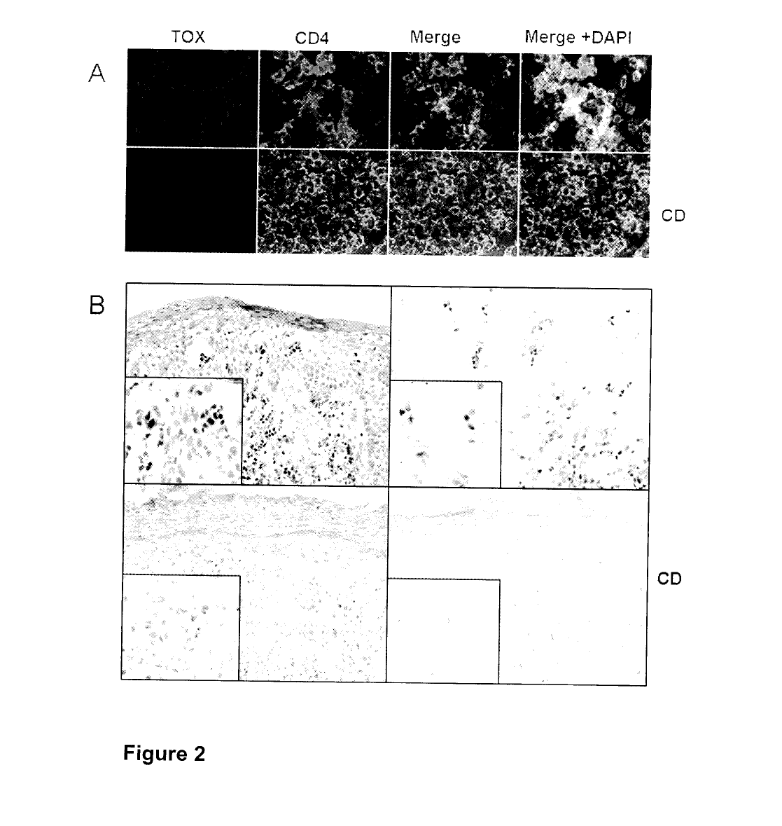

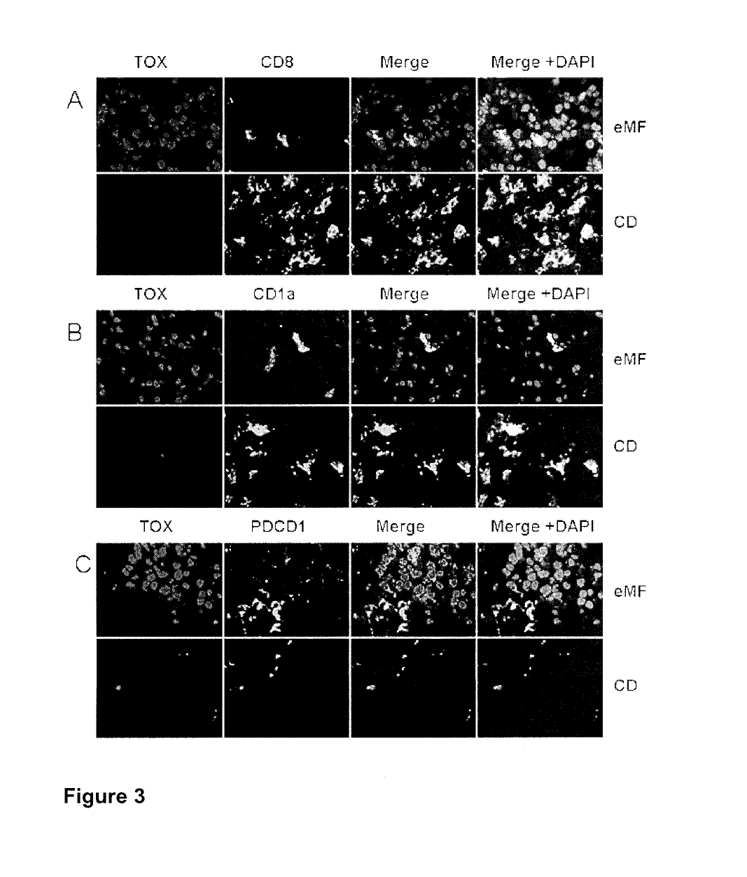

TOX and PDCD1 were further evaluated for their ability to identify CD4+ T cells in eMF biopsies using chronic dermatosis as the controls using immunofluorescence (IF) and immunohistochemistry (IHC). NS contained few CD4 T cells (data not shown). CD biopsies contained variable numbers of CD4 T cells. The vast majority did not show any detectable staining of TOX by IF or IHC, although some (less than 5%) showed dim and focal nuclear staining (FIG. 2, Table 3). In contrast, there was a marked increase of cells with TOX staining in eMF samples. Not only did all 8 eMF samples contained much higher cell numbers with dim focal nuclear TOX staining, all eMF samples also contained 11% to 69% CD4+ T cells demonstrating bright diffused nuclear TOX staining, whereas this pattern of staining was not observed in any of the four CD samples (FIG. 2, Table 3). PDCD1 showed a membrane staining of CD4 T cells in eMF (Table 3, FIG. 3C), although this was not specific to eMF since one of the 4 CD samples showed strong PDCD1 membrane staining.

TOX antibody was further evaluated by immunohistochemistry, a technique available in routine pathology laboratories, for its ability to specifically label cells in eMF biopsies using CD biopsies as the controls. TOX, while showing no significant staining in CD, demonstrated intense staining of cells in eMF skin biopsies, not only in the dermis, but also in the epidermis of eMF samples, including the MF cells in the Pautrier micro-abscess (FIG. 2B).

### Discussion

The diagnosis of early stage MF has been a challenge due to the large variation in clinical manifestations and lack of positive histologic markers. MF is clinically similar to a variety of benign inflammatory skin disorders, such as chronic dermatitis, psoriasis, pigmented purpuric dermatitis, and even vitiligo, often leading to misdiagnosis and delayed diagnosis that occasionally exceeds a decade (Arai, Katayama et al. 1991). The ISCL criteria have described a series of clinical and histopathologic features of eMF (Olsen, Vonderheid et al. 2007). The proposed algorithmic scoring approaches for evaluating eMF provide a degree of diagnostic standardization. However, this approach involves subjective evaluation standards, which largely rely upon the experience of the pathologists, and thus may not be practical in some centers (Furmanczyk, Wolgamot et al. 2010). In a large scale histology study, Massone et al (Massone, Kodama et al. 2005) reported that only 19% MF cases presented Pautrier's microabscesses, and atypical lymphocytes were present only in 9% of cases. Even epidermitropism, a pathognomonic phenomenon in MF, is completely missing in 4% MF cases (Massone, Kodama et al. 2005). Therefore, a positive histological marker for MF cells would be of considerable value in the diagnosis of MF, especially in early stages, when the malignant cells are few in number.

In this eMF centered transcriptome analysis, a two stepped approach was taken to identify genes more specifically enriched in eMF. First, 349 genes were found to be differentially expressed in eMF compared with NS. Most of these genes regulate inflammation and immune activation, including almost all genes previous reported in MF (Shin, Monti et al. 2007; Litvinov, Jones et al. 2010). Together, these genes regulate inflammation and T cell activation pathways, as well as apoptosis, consistent with earlier demonstration that cutaneous T cell lymphomas contained apoptosis abnormalities (Fargnoli, Edelson et al. 1997; De Panfilis 2002; Klemke, Brenner et al. 2009; Wang, Su et al. 2011). However, most of these genes did not appear to be promising diagnostic markers for eMF since the vast majority of them (N=330) were also enriched in CD, which mimics eMF both in clinical appearance and in histological presence of inflammatory cell infiltrates. Therefore, a second step was employed to filter out these genes, leaving 19 genes with specific enrichment in eMF but not in CD when compared with NS. Among these, TOX has emerged as a sensitive and specific marker for eMF biopsies.

While the exact identity of the TOX positive cells in eMF warrants further elucidation, several lines of observation in the current study strongly suggest that they are the MF cells. First, all Pautrier micro-abscesses in the IHC and IF evaluated samples contain TOX+CD4 T cells (FIG. 2); Second, TOX+ cells only represent a subset of CD4+ T cells in the skin biopsies that only came from patients with confirmed eMF diagnosis, with bright diffused nuclear staining not observed in any CD4+ cells from CD biopsies; Third, in the eMF tissues, the cells with TOX staining were the atypical appearing cells, with large and atypical nuclei. TOX antibody did not label the CD4+ cells with small round nuclei in the eMF biopsies. Finally, TOX antibody did not label CD8+ T cells, or cells identified with CD1a. It is worth noting that the eMF samples demonstrating no T cell receptor gene rearrangements also contained TOX+CD4 T cells. It remains to be seen if in these biopsies TCR clonality could be demonstrated in purified TOX+ cells, an issue needing further clarification in the future. It appeared that the cell-based analyses (IF and IHC) demonstrated stronger specificity and sensitivity of TOX than the whole-biopsy based analysis such as RT-PCR in the current study.

Several previous reports demonstrated numerous genes with enriched expression in mycosis fungoides, including CXCR3 (Lu, Duvic et al. 2001), IL15 (Asadullah, Haeussler-Quade et al. 2000; Leroy, Dubois et al. 2001), IL23 (Doherty, Ni et al. 2006), and beta defensin (Gambichler, Skrygan et al. 2007). In addition, Tracey et al (Tracey, Villuendas et al. 2003) reported 27 genes that separated MF from inflammatory dermatoses, and constructed a 6-gene prediction model capable of distinguishing MF and inflammatory disease, including FJX1, Hs. 127160, STAT4, SYNE-1B, TRAF1, and BIRC3. More recently, researchers identified 593 genes with greater than 1.5 fold differential changes in MF, and that these genes were able to divide MF subjects into three distinct clusters that had differential progression outcomes (Shin, Monti et al. 2007; Litvinov, Jones et al. 2010). However, none of these studies primarily focused on early mycosis fungoides. Further, while most of the genes identified by these studies also were found to be enriched in eMF samples the current study, they did not appear to be specific to eMF tissues, since they were also found to be up-regulated in chronic dermatitis (Table 4). In addition, none of the previously identified MF markers have been used on skin histological examination of MF skin biopsies. All 19 genes found to be specifically enriched in eMF in the current study were novel observations. The most significantly up-regulated gene by microarray analysis was the TOX gene, which was confirmed both at the messenger RNA level as well as the cellular level using multiple strategies, including routine immunohistochemistry.

TOX encodes a nuclear protein of the high-mobility group (HMG) family and is highly but transiently expressed in thymic tissue (Wilkinson, Chen et al. 2002). HMG box proteins contain DNA-binding domains that allow them to modify chromatin structure by bending and unwinding DNA backbone (Bustin 1999; Thomas and Travers 2001), and therefore they function as transcription factors. TOX expression has been shown to be strictly regulated in thymocyte differentiation. Upon maturation of CD4+ T cells, however, it is switched off prior to the CD4+ cells exiting the thymus, and is never again expressed to a significant level in mature CD4+ T cells, which is consistent with our finding that all CD4+ cells in benign inflammatory dermatoses do not exhibit TOX staining. Experimentally induced expression of TOX results in a perturbation in lineage commitment due to reduced sensitivity to TCR-mediated signaling (Wilkinson, Chen et al. 2002). As shown in the present Example, eMF infiltrating T cells, including the epidermotropic T cells, express the TOX protein. The timing of aberrant expression of TOX in the development of MF cells is not yet clear. MF cells may re-express TOX extra-thymically or they may never have stopped TOX expression during their development.

Positive diagnostic markers have been identified for eMF by comparing the gene expression profiles of eMF lesions, purified Sezary cells and biopsy proven CTLC skin biopsies with normal CD4+ T cells, healthy skin and benign inflammatory skin diseases, such as chronic dermatitis, using high throughput genomic transcription profiling (cDNA microarrays). Three hundred and forty nine genes (N=349) were differentially expressed in eMF and malignant cutaneous lesions compared with normal skin. These genes belong to pathways associated with inflammation, immune activation and apoptosis regulation. Most of these genes (N=330) also demonstrated significant up-regulation in chronic dermatitis, making them non-ideal markers for eMF. Nineteen genes with specific enrichment in eMF lesions were identified that showed no significant up-regulation in chronic dermatitis (or normal skin). Two of them, TOX, and PDCD1 showed high discrimination power between eMF lesions and biopsies from benign dermatitis by reverse transcription coupled polymerase chain reaction (RT-PCR). Further, in immunohistochemistry and immunofluorescence using antibodies against the TOX and PDCD1 proteins, TOX demonstrated highly specific staining of MF cells in eMF skin biopsies, including the early epidermotropic cells in Pautrier's micro-abscesses. These markers individually and in combination show strong specificity (100%) and high sensitivity (96%) for even early cutaneous T cell lymphomas of the skin versus benign skin conditions. Furthermore, in advanced stages of the T cell malignancy, Sezary syndrome, some of these markers, TOX and PDCD1 in particular, also have high sensitivity and specificity. Patients with higher levels of the TOX marker were also observed to have a much worse prognosis than the patients with lower levels of this marker demonstrating the prognostic utility of this marker eMF-enriched genes, especially TOX are therefore useful as molecular markers for the histological diagnosis of eMF, which currently is a major diagnostic challenge in dermatology.

TABLE 1
Characteristics for subjects with early mycosis fungoides (N = 21)

| Pt | Sex/ | | Disease | | MF Lesion | | Clonal | RTPCR^{##} | |
|---|---|---|---|---|---|---|---|---|---|
| No. | Age | Ethnicity | Duration | | Type | Biopsy Site | TCR | TOX | PDCD1 |
| 1** | M/56 | Chinese | 3 | ys | Patch | Abdomen | + | + | − |
| 2** | M/62 | Chinese | 2 | ys | Patch | Flank | + | + | + |
| 3 | F/62 | Caucasian | 6 | ms | Patch | Abdomen | − | + | + |
| 4 | F/42 | Caucasian | 9 | ys | Patch | R arm | ^{na} | − | − |
| 5 | M/30 | South Asian | 7 | ys | Patch | Back | − | + | − |
| 6 | F/65 | Caucasian | 1 | ms | Thin Plaque | L thigh | + | − | − |
| 7** | M/32 | Caucasian | 1 | yr | Patch | L thigh | − | + | + |
| 8 | M/59 | East-Indian | 8 | ys | Patch | L scapula | ^{na} | + | + |
| 9** | M/46 | East-Indian | 10 | ys | Patch | Back | + | + | + |
| 10 | F/47 | Chinese | 10 | ys | Thin Plaque | L shin | + | + | − |
| 11** | M/43 | East-Indian | 1 | ms | Thin Plaque | L thigh | + | + | + |
| 12 | M/65 | Caucasian | 30 | ys | Patch | L buttock | − | + | + |
| 13 | M/30 | Chinese | 6 | ys | Patch | R buttock | − | + | ^{na} |
| 14 | M/28 | Chinese | 13 | ys | Patch | Back | − | − | ^{na} |
| 15 | M/52 | Chinese | 7 | ys | Thin Plaque | Back | ^{na} | + | ^{na} |
| 16 | F/64 | Chinese | 2 | ys | Patch | Buttock | + | + | ^{na} |
| 17 | F/47 | Chinese | 11 | ys | Thin Plaque | Arm | ^{na} | + | ^{na} |
| 18 | F/48 | Chinese | 5 | ys | Patch | Abdomen | − | − | ^{na} |
| 19 | F/37 | Chinese | 10 | ys | Patch | Buttock | ^{na} | − | ^{na} |
| 20 | M/82 | Chinese | 2 | ys | Patch | Back | ^{na} | + | ^{na} |
| 21 | M/42 | Chinese | 5 | ys | Patch | Abdomen | + | − | ^{na} |

Keys:
**samples used for cDNA microarray analyses;
+ Present;
− Absent;
^{##}Gene transcript abundance higher (+) or lower (−) than the highest of the 15 benign inflammatory dermatosis (BID) samples and 21 normal skin (NS) samples;
^{na}Not available;
ys: years;
ms: months.

TABLE 2
Specifically upregulated genes in eMF compared with CD and NS ^{##}

| | | | | | Expression | |
|---|---|---|---|---|---|---|
| Gene | p-value | p-value | Ratio | Ratio | Ratio | |
| Symbol | (un-corr.)* | (corr.)** | (eMF/NS) | (eMF/BID) | (BID/NS) | Putative Function |
| TOX | 1.65E−06 | 0.0496 | 10.38 | 5.36 | 1.94 | Lymphocyte |
| | | | | | | development |
| PDCD1 | 1.63E−06 | 0.0222 | 8.43 | 4.58 | 1.84 | Apoptosis regulation |
| IL23R | 1.30E−06 | 0.0391 | 7.65 | 3.96 | 1.93 | Immune regulation |
| PYHIN1 | 5.51E−09 | 0.0002 | 7.21 | 3.74 | 1.93 | Signal transduction |
| TAGAP | 1.13E−06 | 0.0340 | 5.25 | 3.12 | 1.68 | Immune regulation |
| SKAP1 | 4.58E−07 | 0.0137 | 6.10 | 3.08 | 1.98 | Signal transduction |
| HLA-DPB2 | 1.13E−06 | 0.0338 | 4.60 | 2.94 | 1.56 | Immune regulation |
| GBP2 | 1.57E−06 | 0.0470 | 4.54 | 2.86 | 1.59 | Signal transduction |
| LY9 | 1.59E−06 | 0.0478 | 5.31 | 2.71 | 1.96 | Immune regulation |
| ETS1 | 1.21E−06 | 0.0363 | 4.41 | 2.59 | 1.70 | Signal transduction |
| AGAP2 | 5.32E−08 | 0.0016 | 4.17 | 2.49 | 1.68 | Signal transduction |
| MGAT4A | 1.62E−06 | 0.0485 | 3.52 | 2.46 | 1.43 | Cell adhesion |
| GNGT2 | 1.06E−06 | 0.0318 | 4.54 | 2.36 | 1.92 | Signal transduction |
| IL18BP | 3.75E−08 | 0.0011 | 4.56 | 2.32 | 1.97 | Immune regulation |
| TNFSF13B | 1.07E−07 | 0.0032 | 3.75 | 2.23 | 1.68 | Immune regulation |
| PSME2 | 3.53E−07 | 0.0106 | 3.12 | 2.11 | 1.48 | Signal transduction |
| IFITM1 | 2.28E−08 | 0.0007 | 3.26 | 2.08 | 1.57 | Immune regulation |
| TNFSF10 | 4.12E−08 | 0.0012 | 3.71 | 2.06 | 1.80 | Immune regulation |
| LAT | 1.00E−06 | 0.0301 | 3.67 | 2.05 | 1.79 | Immune regulation |

^{##}Skin biopsies were prepared from early mycosis fungoides (eMF), benign chronic dermatitis (CD) and normal skin (NS), and placed in RNAlater preservative solution before messenger RNA extraction, and subjected to transcriptome analysis using Agilent whole genome microarrays containing 41,059 unique transcripts. Listed here are genes specifically up-regulated in eMF group compared with both CD and NS. The putative function of each gene is shown in the last column.
*The un-corrected p values represent Volcano plot filtering using Gene Spring software (7.3) using unpaired two tailed t test;
**After Bonferroni correction for multiple testing, the corrected p values were obtained.

TABLE 3
Immunoflurescence detection of TOX and
PDCD1 in eMF and CD ^{# #}

| | % of CD4+ T cells Staining | |
|---|---|---|
| Number | Positive by Immunofluorescence | |
| of CD4+ | | TOX nuclear staining |

| Subject | Cells | Bright | Dim | PDCD1 |
|---|---|---|---|---|
| No. | Scored * | diffused ^{a} | focal ^{b} | Staining ^{c} |
| MF-4 | 133 | 41% | 9% | 0 |
| MF-5 | 60 | 60% | 10% | 7% |
| MF-6 | 140 | 11% | 9% | 5% |
| MF-7 | 175 | 69% | 9% | 24% |
| MF-8 | 160 | 64% | 11% | 35% |
| MF-9 | 99 | 41% | 13% | na |
| MF-11 | 190 | 18% | 13% | 9% |
| MF-12 | 130 | 19% | 15% | 3% |
| CD 1 | 84 | 0 | 0 | 6% |
| CD 2 | 113 | 0 | 3% | 0 |
| CD 3 | 126 | 0 | 5% | 85% |
| CD 4 | 71 | 0 | 3% | 2% |

^{# #} Immune fluorescence staining of 4 micrometer sections were performed according to multi-colored protocol detailed in the text.
* Average number of CD4+ Cells per high power view (average of 3);
^{a} Bright, diffused nuclear staining;
^{b} dim, and focal/dot-like nuclear staining
^{c} cytoplasmic membrane staining;
na Not available

TABLE 4
Genes with differential expression in eMF compared with NS
The expression of 41,059 human transcripts was assessed using Agilent G4112F arrays. All
genes with expression levels >100 in 5/25 samples were analyzed using Gene Spring (7.3)
software. Genes with two fold or more changes (both up or down regulation) with un-paired T
test (Volcano plot) p < 0.05 (Bonferroni corrected for multiple testing) in eMF compared with
normal control skin are listed. The average expression levels in eMF, benign chronic
dermatitis (CD) and normal skin (NS) are also listed, together with the expression ratios
(eMF/CD, and CD/NS). From 486 transcripts, hypothetical genes were removed, whereas the
duplicate transcripts representing the same gene were averaged, leaving 349 genes in total.

| | | | | | | Average | Average | Average |
|---|---|---|---|---|---|---|---|---|
| | | | | | | Signal | Signal | Signal |
| Gene | p-value | p-value | Ratio | Ratio | Ratio | Intensity | Intensity | Intensity |
| Symbol | Corrected | Uncorrected | eMF/NS | eMF/CD | CD/NS | (eMF, N = 5) | (CD, N = 5) | (NS, N = 15) |
| CXCL10 | 0.0283 | 9.45E−07 | 101.11 | 5.15 | 19.64 | 26470 | 5142 | 262 |
| GZMB | 0.0000 | 6.20E−10 | 54.40 | 3.67 | 14.81 | 26402 | 7188 | 485 |
| KIR2DS2 | 0.0016 | 5.20E−08 | 52.55 | 4.11 | 12.78 | 1342 | 326 | 26 |
| APOBEC3A | 0.0319 | 1.06E−06 | 47.06 | 2.63 | 17.87 | 1638 | 622 | 35 |
| IFNG | 0.0118 | 3.95E−07 | 46.01 | 8.56 | 5.38 | 1641 | 192 | 36 |
| CXCL9 | 0.0324 | 1.08E−06 | 45.74 | 5.07 | 9.03 | 84110 | 16595 | 1839 |
| KIR2DS4 | 0.0118 | 3.92E−07 | 36.90 | 4.15 | 8.88 | 996 | 240 | 27 |
| NCR1 | 0.0026 | 8.70E−08 | 34.87 | 6.48 | 5.38 | 867 | 134 | 25 |
| BATF2 | 0.0064 | 2.14E−07 | 33.05 | 5.54 | 5.97 | 5312 | 959 | 161 |
| KLRC3 | 0.0434 | 1.45E−06 | 31.64 | 4.41 | 7.18 | 1076 | 244 | 34 |
| GNLY | 0.0009 | 3.11E−08 | 28.21 | 5.87 | 4.81 | 4253 | 725 | 151 |
| SH2D1A | 0.0090 | 3.01E−07 | 27.91 | 6.79 | 4.11 | 2737 | 403 | 98 |
| GBP5 | 0.0041 | 1.37E−07 | 27.33 | 5.90 | 4.63 | 18921 | 3206 | 692 |
| KIR2DL2 | 0.0151 | 5.03E−07 | 25.89 | 4.88 | 5.30 | 968 | 198 | 37 |
| OAS2 | 0.0024 | 8.13E−08 | 22.62 | 2.22 | 10.18 | 819 | 369 | 36 |
| CLEC4E | 0.0151 | 5.02E−07 | 22.25 | 6.16 | 3.61 | 1860 | 302 | 84 |
| KLHDC7B | 0.0460 | 1.53E−06 | 21.27 | 7.39 | 2.88 | 9714 | 1315 | 457 |
| GBP1 | 0.0004 | 1.19E−08 | 20.86 | 4.85 | 4.30 | 26497 | 5465 | 1270 |
| BCL2L14 | 0.0000 | 4.41E−10 | 20.74 | 3.44 | 6.02 | 883 | 257 | 43 |
| CD247 | 0.0021 | 6.97E−08 | 19.56 | 4.31 | 4.54 | 4827 | 1121 | 247 |
| LAIR2 | 0.0025 | 8.25E−08 | 18.85 | 1.45 | 13.01 | 754 | 520 | 40 |
| ZBTB32 | 0.0000 | 1.03E−09 | 18.58 | 2.34 | 7.93 | 474 | 203 | 26 |
| IDO1 | 0.0039 | 1.29E−07 | 18.45 | 3.03 | 6.10 | 5757 | 1902 | 312 |
| IL2RA | 0.0009 | 2.91E−08 | 18.05 | 1.21 | 14.91 | 1009 | 833 | 56 |
| OASL | 0.0005 | 1.76E−08 | 17.94 | 3.08 | 5.83 | 9366 | 3042 | 522 |
| UBD | 0.0004 | 1.35E−08 | 17.16 | 3.93 | 4.37 | 46471 | 11833 | 2708 |
| OAS2 | 0.0010 | 3.24E−08 | 17.01 | 2.15 | 7.90 | 1654 | 768 | 97 |
| IL12RB2 | 0.0000 | 6.40E−11 | 16.43 | 3.47 | 4.74 | 1638 | 473 | 100 |
| AIM2 | 0.0000 | 9.17E−11 | 16.29 | 3.90 | 4.17 | 1901 | 487 | 117 |
| EPSTI1 | 0.0000 | 3.54E−10 | 15.57 | 3.37 | 4.62 | 3195 | 949 | 205 |
| GPR18 | 0.0218 | 7.26E−07 | 15.00 | 4.26 | 3.52 | 658 | 154 | 44 |
| ARL14 | 0.0003 | 8.72E−09 | 14.69 | 6.81 | 2.16 | 590 | 87 | 40 |
| SLAMF1 | 0.0229 | 7.63E−07 | 14.65 | 2.27 | 6.45 | 2568 | 1130 | 175 |
| SIRPG | 0.0067 | 2.22E−07 | 14.63 | 4.05 | 3.61 | 2780 | 686 | 190 |
| CXCR6 | 0.0063 | 2.09E−07 | 14.16 | 2.15 | 6.60 | 1222 | 569 | 86 |
| FAM26F | 0.0039 | 1.29E−07 | 14.11 | 5.24 | 2.69 | 12599 | 2404 | 893 |
| PRF1 | 0.0037 | 1.22E−07 | 14.04 | 5.36 | 2.62 | 1112 | 207 | 79 |
| JAKMIP1 | 0.0151 | 5.05E−07 | 14.04 | 3.07 | 4.58 | 1466 | 478 | 104 |
| GBP4 | 0.0057 | 1.89E−07 | 13.96 | 4.14 | 3.37 | 10080 | 2435 | 722 |
| TIGIT | 0.0123 | 4.10E−07 | 13.53 | 2.50 | 5.42 | 1456 | 583 | 108 |
| ITK | 0.0325 | 1.08E−06 | 13.27 | 3.86 | 3.44 | 2020 | 523 | 152 |
| HERC6 | 0.0020 | 6.63E−08 | 13.25 | 2.92 | 4.54 | 13015 | 4464 | 982 |
| TBX21 | 0.0058 | 1.94E−07 | 13.12 | 3.04 | 4.31 | 2396 | 788 | 183 |
| LYZ | 0.0193 | 6.43E−07 | 12.61 | 3.49 | 3.61 | 10544 | 3019 | 836 |
| CD38 | 0.0124 | 4.14E−07 | 12.55 | 3.74 | 3.36 | 295 | 79 | 23 |
| MIR155HG | 0.0002 | 5.51E−09 | 12.44 | 3.22 | 3.86 | 470 | 146 | 38 |
| PARP15 | 0.0000 | 4.10E−11 | 12.43 | 3.07 | 4.05 | 273 | 89 | 22 |
| IKZF3 | 0.0470 | 1.57E−06 | 12.40 | 3.03 | 4.09 | 626 | 207 | 51 |
| BCL2A1 | 0.0059 | 1.97E−07 | 12.21 | 3.75 | 3.26 | 1041 | 278 | 85 |
| IFIT3 | 0.0414 | 1.38E−06 | 11.99 | 3.81 | 3.15 | 5573 | 1462 | 465 |
| MX1 | 0.0031 | 1.02E−07 | 11.62 | 2.99 | 3.89 | 58085 | 19453 | 5000 |
| SAMSN1 | 0.0059 | 1.98E−07 | 11.59 | 2.44 | 4.76 | 3897 | 1599 | 336 |
| FASLG | 0.0170 | 5.68E−07 | 11.53 | 3.21 | 3.59 | 1872 | 583 | 162 |
| CD274 | 0.0001 | 4.95E−09 | 11.14 | 2.16 | 5.16 | 3036 | 1406 | 273 |
| CRTAM | 0.0076 | 2.53E−07 | 10.96 | 3.84 | 2.85 | 1021 | 266 | 93 |
| ABCG4 | 0.0219 | 7.31E−07 | 10.95 | 1.87 | 5.85 | 1160 | 620 | 106 |
| IL4I1 | 0.0105 | 3.51E−07 | 10.86 | 1.95 | 5.58 | 11971 | 6154 | 1103 |
| STAT1 | 0.0002 | 6.66E−09 | 10.64 | 2.90 | 3.67 | 19994 | 6892 | 1880 |
| APOBEC3G | 0.0001 | 3.32E−09 | 10.61 | 3.61 | 2.94 | 4424 | 1227 | 417 |
| ICOS | 0.0016 | 5.34E−08 | 10.46 | 1.82 | 5.75 | 1218 | 669 | 116 |
| TFEC | 0.0341 | 1.14E−06 | 10.40 | 1.55 | 6.69 | 363 | 233 | 35 |
| TOX | 0.0496 | 1.65E−06 | 10.38 | 5.36 | 1.94 | 765 | 143 | 74 |
| IRF8 | 0.0029 | 9.58E−08 | 10.33 | 2.30 | 4.49 | 1903 | 827 | 184 |
| PLEK | 0.0017 | 5.68E−08 | 10.05 | 2.40 | 4.18 | 4909 | 2044 | 489 |
| CD2 | 0.0247 | 8.22E−07 | 9.80 | 2.89 | 3.39 | 36977 | 12789 | 3774 |
| OAS3 | 0.0015 | 5.09E−08 | 9.75 | 2.44 | 3.99 | 5491 | 2248 | 563 |
| ZAP70 | 0.0017 | 5.80E−08 | 9.56 | 3.33 | 2.87 | 4588 | 1379 | 480 |
| IL21R | 0.0016 | 5.50E−08 | 9.49 | 3.24 | 2.93 | 2089 | 646 | 220 |
| SAMD3 | 0.0368 | 1.23E−06 | 9.45 | 2.80 | 3.37 | 1199 | 428 | 127 |
| AOAH | 0.0104 | 3.48E−07 | 9.35 | 3.66 | 2.55 | 981 | 268 | 105 |
| TRIM22 | 0.0005 | 1.58E−08 | 9.22 | 2.42 | 3.81 | 9962 | 4117 | 1081 |
| CD27 | 0.0135 | 4.49E−07 | 9.18 | 2.49 | 3.69 | 2985 | 1201 | 325 |
| IL2RG | 0.0122 | 4.07E−07 | 8.84 | 3.08 | 2.87 | 719 | 234 | 81 |
| NLRC3 | 0.0185 | 6.18E−07 | 8.47 | 2.54 | 3.33 | 3707 | 1459 | 438 |
| PDCD1 | 0.0222 | 1.63E−06 | 8.43 | 4.58 | 1.84 | 3131 | 683 | 371 |
| TYMP | 0.0042 | 1.41E−07 | 8.42 | 2.40 | 3.50 | 58017 | 24131 | 6889 |
| CYBB | 0.0327 | 1.09E−06 | 8.15 | 3.17 | 2.58 | 11176 | 3530 | 1371 |
| RTP4 | 0.0071 | 2.38E−07 | 8.14 | 1.84 | 4.43 | 8085 | 4400 | 994 |
| CLEC7A | 0.0012 | 4.06E−08 | 8.11 | 2.66 | 3.05 | 270 | 102 | 33 |
| RGL4 | 0.0275 | 9.17E−07 | 8.09 | 2.33 | 3.47 | 1788 | 766 | 221 |
| HSH2D | 0.0200 | 6.67E−07 | 8.07 | 2.23 | 3.62 | 1112 | 499 | 138 |
| IFI44 | 0.0176 | 5.87E−07 | 7.73 | 2.42 | 3.19 | 15027 | 6208 | 1945 |
| PTPN22 | 0.0033 | 1.09E−07 | 7.71 | 2.24 | 3.44 | 501 | 224 | 65 |
| IL23R | 0.0391 | 1.30E−06 | 7.65 | 3.96 | 1.93 | 245 | 62 | 32 |
| IKZF1 | 0.0195 | 6.51E−07 | 7.55 | 2.36 | 3.20 | 541 | 229 | 72 |
| GPR65 | 0.0441 | 1.47E−06 | 7.55 | 2.61 | 2.89 | 1337 | 512 | 177 |
| JAK3 | 0.0006 | 2.16E−08 | 7.45 | 2.50 | 2.98 | 12756 | 5110 | 1713 |
| DOCK2 | 0.0009 | 2.83E−08 | 7.39 | 2.01 | 3.68 | 1354 | 674 | 183 |
| TRAF3IP3 | 0.0161 | 5.36E−07 | 7.36 | 2.73 | 2.70 | 11926 | 4368 | 1620 |
| PYHIN1 | 0.0002 | 5.51E−09 | 7.21 | 3.74 | 1.93 | 203 | 54 | 28 |
| ERMN | 0.0116 | 3.86E−07 | 7.10 | 2.79 | 2.54 | 303 | 109 | 43 |
| PTPRC | 0.0047 | 1.57E−07 | 7.05 | 2.33 | 3.03 | 8141 | 3501 | 1154 |
| RRM2 | 0.0120 | 3.99E−07 | 6.95 | 1.64 | 4.23 | 1545 | 941 | 222 |
| SRGN | 0.0327 | 1.09E−06 | 6.94 | 1.98 | 3.50 | 19696 | 9924 | 2837 |
| IF127 | 0.0229 | 7.64E−07 | 6.90 | 1.36 | 5.08 | 181619 | 133728 | 26316 |
| LCK | 0.0129 | 4.32E−07 | 6.89 | 2.09 | 3.29 | 4620 | 2208 | 670 |
| BATF | 0.0085 | 2.84E−07 | 6.86 | 2.14 | 3.21 | 15225 | 7116 | 2218 |
| CTLA4 | 0.0000 | 1.08E−12 | 6.82 | 1.45 | 4.69 | 135 | 93 | 20 |
| SAMD9 | 0.0033 | 1.09E−07 | 6.82 | 2.75 | 2.48 | 7654 | 2781 | 1123 |
| CD53 | 0.0013 | 4.34E−08 | 6.80 | 1.99 | 3.41 | 8971 | 4506 | 1320 |
| LCP2 | 0.0121 | 4.04E−07 | 6.77 | 2.05 | 3.30 | 6517 | 3181 | 963 |
| CARD11 | 0.0312 | 1.04E−06 | 6.72 | 2.70 | 2.49 | 469 | 174 | 70 |
| GVINP1 | 0.0113 | 3.76E−07 | 6.56 | 2.47 | 2.65 | 668 | 270 | 102 |
| MX2 | 0.0026 | 8.56E−08 | 6.53 | 3.06 | 2.13 | 10830 | 3539 | 1658 |
| SAMD9L | 0.0007 | 2.25E−08 | 6.52 | 1.85 | 3.52 | 5402 | 2921 | 829 |
| BIRC3 | 0.0433 | 1.44E−06 | 6.41 | 1.56 | 4.11 | 11422 | 7326 | 1782 |
| RASAL3 | 0.0024 | 8.04E−08 | 6.31 | 2.79 | 2.26 | 13224 | 4744 | 2095 |
| MEI1 | 0.0028 | 9.38E−08 | 6.28 | 2.24 | 2.80 | 5619 | 2509 | 895 |
| IFIH1 | 0.0100 | 3.34E−07 | 6.24 | 2.44 | 2.56 | 12420 | 5099 | 1989 |
| SLAMF7 | 0.0028 | 9.22E−08 | 6.19 | 2.24 | 2.76 | 5478 | 2443 | 886 |
| SLAMF8 | 0.0010 | 3.48E−08 | 6.17 | 1.97 | 3.14 | 10253 | 5207 | 1661 |
| BIN2 | 0.0175 | 5.84E−07 | 6.16 | 1.94 | 3.17 | 1483 | 763 | 241 |
| P2RY8 | 0.0203 | 6.77E−07 | 6.16 | 2.10 | 2.93 | 1415 | 674 | 230 |
| USP18 | 0.0317 | 1.06E−06 | 6.13 | 2.10 | 2.92 | 7437 | 3539 | 1213 |
| SKAP1 | 0.0137 | 4.58E−07 | 6.10 | 3.08 | 1.98 | 8519 | 2768 | 1398 |
| SNX10 | 0.0004 | 1.46E−08 | 6.07 | 1.73 | 3.51 | 8671 | 5017 | 1428 |
| APOL6 | 0.0001 | 3.28E−09 | 6.06 | 2.10 | 2.88 | 3166 | 1506 | 522 |
| CSF2RA | 0.0052 | 1.74E−07 | 6.06 | 1.68 | 3.60 | 1048 | 623 | 173 |
| ITGAX | 0.0060 | 1.99E−07 | 6.02 | 1.49 | 4.04 | 238 | 160 | 40 |
| PARP9 | 0.0031 | 1.04E−07 | 6.02 | 2.10 | 2.86 | 11374 | 5406 | 1890 |
| SASH3 | 0.0355 | 1.19E−06 | 5.99 | 2.13 | 2.82 | 2950 | 1387 | 492 |
| APOL1 | 0.0351 | 1.17E−06 | 5.98 | 2.84 | 2.11 | 833 | 294 | 139 |
| RCSD1 | 0.0026 | 8.72E−08 | 5.90 | 2.61 | 2.26 | 4281 | 1637 | 725 |
| ITGB2 | 0.0134 | 4.48E−07 | 5.89 | 2.01 | 2.94 | 16133 | 8041 | 2738 |
| DOCK8 | 0.0406 | 1.35E−06 | 5.85 | 2.34 | 2.50 | 258 | 110 | 44 |
| C1QC | 0.0433 | 1.44E−06 | 5.82 | 1.31 | 4.44 | 1781 | 1358 | 306 |
| LILRB3 | 0.0095 | 3.18E−07 | 5.77 | 1.54 | 3.75 | 1256 | 816 | 218 |
| LIMD2 | 0.0017 | 5.61E−08 | 5.66 | 2.37 | 2.39 | 1927 | 814 | 340 |
| SLCO2B1 | 0.0082 | 2.74E−07 | 5.49 | 1.77 | 3.10 | 10216 | 5767 | 1862 |
| HAPLN3 | 0.0011 | 3.72E−08 | 5.46 | 2.61 | 2.09 | 433 | 166 | 79 |
| CHST11 | 0.0263 | 8.77E−07 | 5.32 | 2.30 | 2.31 | 1236 | 537 | 232 |
| LY9 | 0.0478 | 1.59E−06 | 5.31 | 2.71 | 1.96 | 452 | 167 | 85 |
| COTL1 | 0.0002 | 7.91E−09 | 5.29 | 1.92 | 2.75 | 44347 | 23095 | 8388 |
| ARHGAP15 | 0.0031 | 1.02E−07 | 5.27 | 2.45 | 2.15 | 1953 | 796 | 370 |
| TAGAP | 0.0340 | 1.13E−06 | 5.25 | 3.12 | 1.68 | 1831 | 586 | 349 |
| PIM2 | 0.0103 | 3.42E−07 | 5.21 | 2.58 | 2.02 | 3118 | 1209 | 599 |
| IRF1 | 0.0500 | 1.67E−06 | 5.18 | 2.48 | 2.09 | 4819 | 1941 | 930 |
| APOL3 | 0.0003 | 1.01E−08 | 5.15 | 2.42 | 2.13 | 17920 | 7403 | 3478 |
| MNDA | 0.0093 | 3.11E−07 | 5.05 | 2.09 | 2.42 | 5220 | 2495 | 1033 |
| ISG20 | 0.0079 | 2.64E−07 | 5.04 | 2.14 | 2.35 | 39097 | 18265 | 7762 |
| SOD2 | 0.0265 | 8.84E−07 | 4.99 | 2.16 | 2.31 | 16629 | 7684 | 3332 |
| KLRD1 | 0.0093 | 3.10E−07 | 4.96 | 2.17 | 2.29 | 120 | 55 | 24 |
| FAM78A | 0.0066 | 2.19E−07 | 4.95 | 1.92 | 2.58 | 3243 | 1690 | 656 |
| GIMAP2 | 0.0018 | 5.84E−08 | 4.94 | 2.39 | 2.07 | 3844 | 1609 | 779 |
| DDX60L | 0.0112 | 3.73E−07 | 4.87 | 2.11 | 2.31 | 2715 | 1289 | 558 |
| C1orf38 | 0.0421 | 1.40E−06 | 4.86 | 1.43 | 3.41 | 10776 | 7556 | 2216 |
| HERC5 | 0.0000 | 1.06E−09 | 4.83 | 2.35 | 2.05 | 955 | 406 | 198 |
| IRF7 | 0.0005 | 1.77E−08 | 4.82 | 1.56 | 3.09 | 16080 | 10302 | 3334 |
| PSMB9 | 0.0020 | 6.66E−08 | 4.82 | 2.01 | 2.40 | 29559 | 14715 | 6129 |
| CYTIP | 0.0020 | 6.68E−08 | 4.82 | 1.80 | 2.69 | 6000 | 3343 | 1244 |
| KIF21B | 0.0193 | 6.45E−07 | 4.81 | 2.28 | 2.11 | 939 | 412 | 195 |
| ADAM8 | 0.0429 | 1.43E−06 | 4.76 | 1.42 | 3.35 | 26240 | 18436 | 5511 |
| HAVCR2 | 0.0028 | 9.40E−08 | 4.73 | 1.14 | 4.17 | 5738 | 5054 | 1212 |
| FERMT3 | 0.0052 | 1.73E−07 | 4.68 | 1.53 | 3.06 | 1436 | 938 | 307 |
| RHOH | 0.0046 | 1.55E−07 | 4.68 | 1.97 | 2.37 | 1092 | 554 | 234 |
| WIPF1 | 0.0015 | 5.08E−08 | 4.68 | 1.93 | 2.42 | 2586 | 1339 | 553 |
| CYTH4 | 0.0197 | 6.56E−07 | 4.67 | 2.09 | 2.23 | 312 | 149 | 67 |
| SERPINB9 | 0.0077 | 2.57E−07 | 4.61 | 2.29 | 2.01 | 3807 | 1662 | 826 |
| HLA-DPB2 | 0.0338 | 1.13E−06 | 4.60 | 2.94 | 1.56 | 31295 | 10634 | 6802 |
| SAMHD1 | 0.0087 | 2.89E−07 | 4.56 | 1.70 | 2.69 | 450 | 265 | 99 |
| IL18BP | 0.0011 | 3.75E−08 | 4.56 | 2.32 | 1.97 | 1505 | 649 | 330 |
| GNGT2 | 0.0318 | 1.06E−06 | 4.54 | 2.36 | 1.92 | 219 | 93 | 48 |
| GBP2 | 0.0470 | 1.57E−06 | 4.54 | 2.86 | 1.59 | 27363 | 9562 | 6029 |
| ARHGAP9 | 0.0155 | 5.15E−07 | 4.54 | 1.62 | 2.80 | 5761 | 3559 | 1270 |
| APOL2 | 0.0027 | 8.93E−08 | 4.53 | 1.92 | 2.36 | 18515 | 9665 | 4089 |
| FMNL1 | 0.0014 | 4.77E−08 | 4.50 | 2.07 | 2.17 | 774 | 373 | 172 |
| PARP8 | 0.0305 | 1.02E−06 | 4.48 | 1.54 | 2.91 | 1742 | 1133 | 389 |
| ADORA2A | 0.0087 | 2.91E−07 | 4.47 | 2.12 | 2.11 | 1122 | 530 | 251 |
| TNFRSF6B | 0.0044 | 1.48E−07 | 4.46 | 1.68 | 2.66 | 3215 | 1918 | 722 |
| SNX20 | 0.0081 | 2.69E−07 | 4.44 | 1.66 | 2.67 | 7783 | 4691 | 1755 |
| INSL3 | 0.0395 | 1.32E−06 | 4.44 | 1.78 | 2.49 | 396 | 223 | 89 |
| SLA | 0.0364 | 1.21E−06 | 4.42 | 2.01 | 2.20 | 21942 | 10933 | 4969 |
| ETS1 | 0.0363 | 1.21E−06 | 4.41 | 2.59 | 1.70 | 432 | 167 | 98 |
| ICAM1 | 0.0031 | 1.02E−07 | 4.38 | 1.59 | 2.75 | 2952 | 1851 | 673 |
| MELK | 0.0061 | 2.03E−07 | 4.37 | 1.30 | 3.36 | 1330 | 1023 | 304 |
| LCP1 | 0.0007 | 2.49E−08 | 4.32 | 2.11 | 2.05 | 56206 | 26690 | 13005 |
| DOCK10 | 0.0010 | 3.24E−08 | 4.32 | 1.62 | 2.67 | 5502 | 3405 | 1275 |
| TRAF1 | 0.0052 | 1.72E−07 | 4.29 | 1.74 | 2.47 | 2201 | 1268 | 513 |
| CD86 | 0.0017 | 5.55E−08 | 4.23 | 1.48 | 2.86 | 1196 | 809 | 283 |
| CD74 | 0.0076 | 2.55E−07 | 4.17 | 2.09 | 2.00 | 8097 | 3871 | 1940 |
| RASSF4 | 0.0471 | 1.57E−06 | 4.17 | 1.87 | 2.23 | 4123 | 2200 | 989 |
| AGAP2 | 0.0016 | 5.32E−08 | 4.17 | 2.49 | 1.68 | 1854 | 746 | 445 |
| IFI30 | 0.0088 | 2.93E−07 | 4.13 | 1.68 | 2.46 | 58923 | 35048 | 14271 |
| PTPRJ | 0.0008 | 2.60E−08 | 4.13 | 1.79 | 2.31 | 4723 | 2638 | 1144 |
| BLM | 0.0002 | 7.08E−09 | 4.11 | 1.74 | 2.37 | 1688 | 972 | 410 |
| LPXN | 0.0074 | 2.48E−07 | 4.11 | 1.98 | 2.07 | 2793 | 1410 | 680 |
| NMI | 0.0022 | 7.29E−08 | 4.01 | 1.67 | 2.41 | 11574 | 6943 | 2883 |
| PREX1 | 0.0026 | 8.68E−08 | 3.97 | 1.88 | 2.11 | 7944 | 4228 | 2000 |
| SP110 | 0.0181 | 6.05E−07 | 3.95 | 1.79 | 2.21 | 5362 | 2992 | 1356 |
| DENND1C | 0.0102 | 3.41E−07 | 3.94 | 2.04 | 1.93 | 1320 | 647 | 335 |
| LAMP3 | 0.0142 | 4.75E−07 | 3.94 | 1.36 | 2.90 | 16816 | 12395 | 4269 |
| HCLS1 | 0.0049 | 1.63E−07 | 3.92 | 1.74 | 2.26 | 32705 | 18830 | 8345 |
| TLR6 | 0.0280 | 9.34E−07 | 3.86 | 1.85 | 2.08 | 848 | 457 | 219 |
| IL15 | 0.0252 | 8.39E−07 | 3.78 | 1.57 | 2.41 | 6793 | 4331 | 1797 |
| BTN3A3 | 0.0251 | 8.35E−07 | 3.77 | 1.57 | 2.40 | 2767 | 1759 | 734 |
| TNFSF13B | 0.0032 | 1.07E−07 | 3.75 | 2.23 | 1.68 | 3606 | 1614 | 963 |
| ZC3HAV1 | 0.0009 | 2.91E−08 | 3.73 | 2.03 | 1.83 | 1387 | 682 | 372 |
| TNFSF10 | 0.0012 | 4.12E−08 | 3.71 | 2.06 | 1.80 | 20218 | 9797 | 5444 |
| LAT | 0.0301 | 1.00E−06 | 3.67 | 2.05 | 1.79 | 14697 | 7161 | 4001 |
| FGR | 0.0440 | 1.47E−06 | 3.67 | 1.18 | 3.11 | 9793 | 8300 | 2666 |
| CDH3 | 0.0294 | 9.82E−07 | 3.67 | 1.57 | 2.33 | 15210 | 9664 | 4142 |
| NCAPG | 0.0099 | 3.31E−07 | 3.67 | 1.41 | 2.60 | 5407 | 3833 | 1473 |
| NDC80 | 0.0154 | 5.12E−07 | 3.67 | 1.64 | 2.24 | 4838 | 2947 | 1318 |
| DTX3L | 0.0082 | 2.74E−07 | 3.64 | 1.44 | 2.54 | 574 | 400 | 158 |
| HLA-DPA1 | 0.0147 | 4.91E−07 | 3.63 | 1.73 | 2.10 | 83543 | 48350 | 23003 |
| AKR1B1 | 0.0290 | 9.66E−07 | 3.60 | 1.55 | 2.32 | 11223 | 7238 | 3122 |
| SEL1L3 | 0.0271 | 9.02E−07 | 3.58 | 1.59 | 2.26 | 4937 | 3110 | 1378 |
| DOK3 | 0.0066 | 2.19E−07 | 3.58 | 1.98 | 1.81 | 1903 | 961 | 531 |
| MYO5A | 0.0001 | 1.95E−09 | 3.54 | 1.28 | 2.78 | 3274 | 2565 | 924 |
| FAM49B | 0.0000 | 1.14E−09 | 3.53 | 1.47 | 2.40 | 13849 | 9390 | 3920 |
| ENTPD1 | 0.0472 | 1.57E−06 | 3.53 | 0.99 | 3.55 | 608 | 611 | 172 |
| MGAT4A | 0.0485 | 1.62E−06 | 3.52 | 2.46 | 1.43 | 2758 | 1119 | 784 |
| MICB | 0.0273 | 9.10E−07 | 3.48 | 1.50 | 2.31 | 1750 | 1164 | 503 |
| P2RY6 | 0.0023 | 7.65E−08 | 3.47 | 1.37 | 2.53 | 577 | 421 | 166 |
| CCRL2 | 0.0120 | 3.98E−07 | 3.46 | 1.69 | 2.05 | 613 | 363 | 177 |
| HLA-DRA | 0.0224 | 7.48E−07 | 3.44 | 1.63 | 2.11 | 90447 | 55487 | 26279 |
| LGALS9C | 0.0125 | 4.15E−07 | 3.44 | 1.29 | 2.67 | 24253 | 18815 | 7051 |
| PGK1 | 0.0140 | 4.68E−07 | 3.44 | 1.71 | 2.01 | 2945 | 1721 | 856 |
| WAS | 0.0368 | 1.23E−06 | 3.37 | 1.57 | 2.15 | 403 | 258 | 120 |
| IGSF6 | 0.0299 | 9.96E−07 | 3.33 | 1.10 | 3.04 | 1032 | 942 | 310 |
| CLEC10A | 0.0150 | 5.01E−07 | 3.30 | 0.93 | 3.57 | 742 | 801 | 225 |
| CHEK1 | 0.0253 | 8.44E−07 | 3.29 | 1.53 | 2.15 | 1410 | 921 | 428 |
| IFITM1 | 0.0007 | 2.28E−08 | 3.26 | 2.08 | 1.57 | 63083 | 30338 | 19321 |
| CCDC57 | 0.0003 | 9.68E−09 | 3.26 | 1.87 | 1.74 | 492 | 263 | 151 |
| TRERF1 | 0.0068 | 2.28E−07 | 3.24 | 1.33 | 2.44 | 1366 | 1031 | 422 |
| ACSL4 | 0.0245 | 8.18E−07 | 3.24 | 1.45 | 2.23 | 2110 | 1456 | 652 |
| CLIC2 | 0.0253 | 8.45E−07 | 3.23 | 1.00 | 3.23 | 1605 | 1608 | 497 |
| SLFN12 | 0.0079 | 2.63E−07 | 3.22 | 1.79 | 1.80 | 555 | 310 | 172 |
| PARP12 | 0.0070 | 2.35E−07 | 3.20 | 1.54 | 2.07 | 7295 | 4727 | 2282 |
| WHSC1 | 0.0003 | 9.97E−09 | 3.13 | 1.54 | 2.03 | 1759 | 1141 | 563 |
| PSME2 | 0.0106 | 3.53E−07 | 3.12 | 2.11 | 1.48 | 162639 | 77162 | 52067 |
| MLKL | 0.0251 | 8.35E−07 | 3.12 | 1.68 | 1.85 | 4267 | 2538 | 1370 |
| FAM105A | 0.0032 | 1.08E−07 | 3.12 | 1.48 | 2.11 | 1793 | 1213 | 575 |
| DRAM1 | 0.0101 | 3.38E−07 | 3.09 | 1.58 | 1.96 | 6619 | 4194 | 2142 |
| SLC20A1 | 0.0053 | 1.75E−07 | 3.03 | 1.69 | 1.79 | 17011 | 10081 | 5622 |
| ARHGDIB | 0.0238 | 7.93E−07 | 3.02 | 1.59 | 1.91 | 45649 | 28790 | 15101 |
| ADCY7 | 0.0142 | 4.73E−07 | 3.00 | 1.24 | 2.41 | 6943 | 5592 | 2317 |
| IFI16 | 0.0000 | 1.16E−09 | 3.00 | 1.27 | 2.36 | 13375 | 10553 | 4465 |
| PDPN | 0.0430 | 1.43E−06 | 2.95 | 1.41 | 2.09 | 2536 | 1795 | 859 |
| IRF9 | 0.0051 | 1.70E−07 | 2.94 | 1.60 | 1.84 | 8535 | 5339 | 2900 |
| SKA2 | 0.0061 | 2.03E−07 | 2.92 | 1.31 | 2.23 | 317 | 242 | 109 |
| ACTR2 | 0.0450 | 1.50E−06 | 2.83 | 1.82 | 1.56 | 8621 | 4749 | 3049 |
| MND1 | 0.0300 | 1.00E−06 | 2.82 | 1.36 | 2.07 | 853 | 628 | 303 |
| RFX5 | 0.0005 | 1.73E−08 | 2.77 | 1.36 | 2.04 | 2617 | 1925 | 945 |
| TIMELESS | 0.0173 | 5.77E−07 | 2.77 | 1.62 | 1.71 | 8294 | 5125 | 2997 |
| PLSCR1 | 0.0200 | 6.68E−07 | 2.76 | 1.34 | 2.07 | 7568 | 5666 | 2740 |
| TRIM14 | 0.0022 | 7.18E−08 | 2.72 | 1.11 | 2.45 | 2129 | 1920 | 784 |
| TPMT | 0.0103 | 3.44E−07 | 2.71 | 1.58 | 1.71 | 2686 | 1696 | 991 |
| PHTF2 | 0.0266 | 8.87E−07 | 2.69 | 1.29 | 2.08 | 3156 | 2441 | 1174 |
| SEC22C | 0.0077 | 2.57E−07 | 2.67 | 1.71 | 1.56 | 305 | 178 | 114 |
| NOD2 | 0.0000 | 8.58E−10 | 2.63 | 1.03 | 2.55 | 6037 | 5866 | 2296 |
| SYNCRIP | 0.0110 | 3.67E−07 | 2.62 | 1.44 | 1.82 | 10355 | 7206 | 3949 |
| ARL11 | 0.0052 | 1.73E−07 | 2.58 | 1.32 | 1.96 | 256 | 194 | 99 |
| ACP5 | 0.0321 | 1.07E−06 | 2.52 | 1.20 | 2.10 | 48990 | 40688 | 19419 |
| CASP10 | 0.0348 | 1.16E−06 | 2.49 | 1.53 | 1.62 | 2580 | 1681 | 1036 |
| IFNGR1 | 0.0001 | 3.60E−09 | 2.46 | 1.46 | 1.69 | 34512 | 23677 | 14045 |
| PCNX | 0.0025 | 8.37E−08 | 2.42 | 1.72 | 1.41 | 1909 | 1110 | 787 |
| PRR13 | 0.0337 | 1.12E−06 | 2.42 | 1.57 | 1.54 | 2688 | 1715 | 1112 |
| TPM3 | 0.0162 | 5.39E−07 | 2.41 | 1.58 | 1.53 | 11149 | 7058 | 4622 |
| TEP1 | 0.0415 | 1.38E−06 | 2.41 | 1.51 | 1.59 | 3555 | 2351 | 1475 |
| NASP | 0.0490 | 1.63E−06 | 2.40 | 1.51 | 1.59 | 7587 | 5032 | 3162 |
| IKZF4 | 0.0003 | 8.93E−09 | 2.39 | 1.48 | 1.61 | 1224 | 827 | 513 |
| NFKBIE | 0.0480 | 1.60E−06 | 2.38 | 1.29 | 1.84 | 6851 | 5293 | 2875 |
| RAN | 0.0076 | 2.52E−07 | 2.38 | 1.53 | 1.56 | 12320 | 8075 | 5184 |
| ME2 | 0.0163 | 5.45E−07 | 2.37 | 1.26 | 1.89 | 1379 | 1096 | 581 |
| TM6SF1 | 0.0061 | 2.03E−07 | 2.37 | 1.20 | 1.98 | 456 | 380 | 192 |
| ACOT9 | 0.0131 | 4.37E−07 | 2.29 | 1.39 | 1.64 | 1584 | 1137 | 692 |
| C12orf35 | 0.0237 | 7.92E−07 | 2.27 | 1.51 | 1.50 | 15226 | 10103 | 6714 |
| DENND1B | 0.0487 | 1.63E−06 | 2.27 | 1.27 | 1.78 | 1551 | 1219 | 684 |
| GRB2 | 0.0137 | 4.58E−07 | 2.26 | 1.54 | 1.47 | 1094 | 713 | 485 |
| PARP11 | 0.0010 | 3.21E−08 | 2.26 | 1.86 | 1.21 | 202 | 108 | 89 |
| ZNF562 | 0.0071 | 2.37E−07 | 2.25 | 0.93 | 2.43 | 840 | 908 | 374 |
| TRIM34 | 0.0135 | 4.51E−07 | 2.20 | 1.52 | 1.45 | 675 | 445 | 306 |
| PPT1 | 0.0017 | 5.65E−08 | 2.20 | 1.18 | 1.86 | 47103 | 39793 | 21406 |
| MCM6 | 0.0148 | 4.94E−07 | 2.17 | 1.51 | 1.43 | 13544 | 8945 | 6245 |
| ARPC3 | 0.0444 | 1.48E−06 | 2.16 | 1.22 | 1.77 | 82019 | 67188 | 38037 |
| GTF3C6 | 0.0016 | 5.23E−08 | 2.14 | 1.30 | 1.65 | 25547 | 19687 | 11915 |
| TMEM206 | 0.0234 | 7.79E−07 | 2.14 | 1.15 | 1.86 | 1060 | 923 | 495 |
| H2AFV | 0.0244 | 8.14E−07 | 2.09 | 1.36 | 1.53 | 35934 | 26335 | 17164 |
| CPSF2 | 0.0314 | 1.05E−06 | 2.07 | 1.30 | 1.60 | 2477 | 1908 | 1196 |
| FAM21C | 0.0359 | 1.20E−06 | 2.03 | 1.30 | 1.57 | 3617 | 2792 | 1783 |
| CTSF | 0.0036 | 1.21E−07 | 0.49 | 0.66 | 0.73 | 12018 | 18078 | 24613 |
| ZFYVE21 | 0.0013 | 4.17E−08 | 0.47 | 0.71 | 0.66 | 14853 | 20886 | 31503 |
| SLC44A2 | 0.0012 | 3.90E−08 | 0.47 | 0.82 | 0.57 | 4316 | 5259 | 9156 |
| C7orf41 | 0.0380 | 1.27E−06 | 0.45 | 0.82 | 0.55 | 5602 | 6844 | 12368 |
| DCBLD2 | 0.0296 | 9.86E−07 | 0.45 | 0.69 | 0.65 | 143 | 207 | 319 |
| LRP6 | 0.0009 | 3.01E−08 | 0.45 | 0.84 | 0.53 | 182 | 217 | 407 |
| ANG | 0.0043 | 1.43E−07 | 0.44 | 0.56 | 0.78 | 3829 | 6828 | 8727 |
| PCDHB14 | 0.0077 | 2.58E−07 | 0.43 | 0.71 | 0.61 | 435 | 613 | 1006 |
| GAS5 | 0.0092 | 3.07E−07 | 0.43 | 0.69 | 0.63 | 6298 | 9193 | 14676 |
| FBXO17 | 0.0074 | 2.48E−07 | 0.43 | 0.71 | 0.60 | 341 | 481 | 800 |
| TNPO2 | 0.0355 | 1.18E−06 | 0.42 | 0.64 | 0.66 | 188 | 296 | 450 |
| MTF1 | 0.0009 | 2.84E−08 | 0.41 | 0.59 | 0.70 | 4384 | 7372 | 10565 |
| SBNO1 | 0.0104 | 3.47E−07 | 0.41 | 0.77 | 0.54 | 1348 | 1759 | 3288 |
| THRA | 0.0156 | 5.19E−07 | 0.40 | 0.87 | 0.46 | 329 | 378 | 815 |
| HIP1 | 0.0389 | 1.30E−06 | 0.40 | 0.60 | 0.67 | 250 | 416 | 619 |
| PTPLAD1 | 0.0000 | 1.19E−10 | 0.40 | 0.69 | 0.58 | 4706 | 6804 | 11645 |
| TTC3 | 0.0025 | 8.23E−08 | 0.40 | 0.69 | 0.58 | 4263 | 6208 | 10665 |
| CEP68 | 0.0155 | 5.18E−07 | 0.40 | 0.72 | 0.56 | 2247 | 3123 | 5625 |
| RPS9 | 0.0064 | 2.12E−07 | 0.40 | 0.59 | 0.67 | 587 | 999 | 1482 |
| MMAB | 0.0249 | 8.30E−07 | 0.40 | 0.78 | 0.51 | 431 | 552 | 1090 |
| C11orf52 | 0.0462 | 1.54E−06 | 0.38 | 0.60 | 0.63 | 595 | 985 | 1554 |
| PTPN11 | 0.0414 | 1.38E−06 | 0.38 | 0.61 | 0.62 | 175 | 286 | 462 |
| NFIX | 0.0255 | 8.49E−07 | 0.38 | 0.77 | 0.49 | 9457 | 12301 | 25179 |
| KDM4A | 0.0421 | 1.40E−06 | 0.37 | 0.61 | 0.61 | 987 | 1613 | 2637 |
| ST6GALNAC2 | 0.0005 | 1.56E−08 | 0.37 | 0.72 | 0.52 | 218 | 304 | 583 |
| ALDH3A2 | 0.0484 | 1.61E−06 | 0.37 | 0.63 | 0.59 | 3920 | 6228 | 10635 |
| C5orf24 | 0.0397 | 1.32E−06 | 0.37 | 0.61 | 0.60 | 2544 | 4172 | 6904 |
| CMTM4 | 0.0000 | 1.59E−09 | 0.37 | 0.74 | 0.50 | 2923 | 3933 | 7943 |
| USP54 | 0.0054 | 1.80E−07 | 0.37 | 0.71 | 0.52 | 3280 | 4626 | 8940 |
| RASL10B | 0.0018 | 5.95E−08 | 0.36 | 0.52 | 0.70 | 33 | 64 | 91 |
| DUX4 | 0.0421 | 1.40E−06 | 0.36 | 0.49 | 0.73 | 37984 | 77271 | 105306 |
| PLEKHM3 | 0.0039 | 1.30E−07 | 0.36 | 0.49 | 0.73 | 285 | 582 | 797 |
| IRS2 | 0.0116 | 3.87E−07 | 0.35 | 0.64 | 0.55 | 2600 | 4056 | 7329 |
| AMOT | 0.0411 | 1.37E−06 | 0.35 | 0.60 | 0.58 | 333 | 552 | 952 |
| YBX2 | 0.0088 | 2.92E−07 | 0.34 | 0.61 | 0.56 | 322 | 532 | 942 |
| ZDHHC9 | 0.0005 | 1.69E−08 | 0.34 | 0.72 | 0.47 | 1854 | 2582 | 5505 |
| EDA | 0.0006 | 1.95E−08 | 0.33 | 0.66 | 0.50 | 286 | 436 | 876 |
| DGAT2 | 0.0229 | 7.64E−07 | 0.32 | 0.61 | 0.53 | 5357 | 8747 | 16603 |
| DLX3 | 0.0436 | 1.45E−06 | 0.32 | 0.63 | 0.51 | 501 | 800 | 1559 |
| DNAH11 | 0.0212 | 7.07E−07 | 0.31 | 0.57 | 0.54 | 131 | 228 | 426 |
| MAP4K5 | 0.0000 | 1.35E−09 | 0.29 | 0.53 | 0.55 | 1171 | 2212 | 4001 |
| ASH1L | 0.0454 | 1.51E−06 | 0.28 | 0.52 | 0.53 | 1200 | 2302 | 4359 |
| ZNRF3 | 0.0449 | 1.50E−06 | 0.27 | 0.53 | 0.51 | 1252 | 2345 | 4581 |
| COQ9 | 0.0030 | 9.87E−08 | 0.26 | 0.43 | 0.61 | 93 | 215 | 352 |
| LIG3 | 0.0228 | 7.59E−07 | 0.25 | 0.49 | 0.52 | 43 | 89 | 171 |
| PPME1 | 0.0338 | 1.13E−06 | 0.25 | 0.52 | 0.49 | 225 | 431 | 884 |
| FP588 | 0.0142 | 4.75E−07 | 0.25 | 0.51 | 0.49 | 43 | 84 | 169 |
| PRKAB2 | 0.0043 | 1.45E−07 | 0.25 | 0.49 | 0.51 | 533 | 1083 | 2130 |
| MACROD2 | 0.0442 | 1.47E−06 | 0.25 | 0.62 | 0.40 | 185 | 297 | 749 |
| SUSD2 | 0.0295 | 9.82E−07 | 0.23 | 0.42 | 0.56 | 1390 | 3322 | 5955 |
| ZC3H7B | 0.0278 | 9.27E−07 | 0.23 | 0.38 | 0.60 | 50 | 132 | 221 |
| TANC2 | 0.0058 | 1.93E−07 | 0.22 | 0.44 | 0.51 | 61 | 140 | 273 |
| UTP14A | 0.0311 | 1.04E−06 | 0.22 | 0.41 | 0.54 | 343 | 833 | 1557 |
| GPR150 | 0.0345 | 1.15E−06 | 0.22 | 0.34 | 0.63 | 7441 | 21634 | 34305 |
| C9orf131 | 0.0037 | 1.22E−07 | 0.22 | 0.65 | 0.33 | 50 | 77 | 232 |
| ZSCAN18 | 0.0198 | 6.60E−07 | 0.21 | 0.38 | 0.55 | 253 | 662 | 1194 |
| TET2 | 0.0487 | 1.62E−06 | 0.20 | 0.34 | 0.59 | 187 | 543 | 928 |
| LIPH | 0.0035 | 1.17E−07 | 0.17 | 0.34 | 0.50 | 32 | 94 | 187 |
| EDIL3 | 0.0228 | 7.59E−07 | 0.17 | 0.53 | 0.32 | 61 | 116 | 363 |
| ADAMTSL3 | 0.0331 | 1.10E−06 | 0.15 | 0.40 | 0.38 | 150 | 373 | 969 |
| ACADL | 0.0002 | 8.18E−09 | 0.15 | 0.50 | 0.31 | 25 | 51 | 166 |
| OTX1 | 0.0013 | 4.29E−08 | 0.14 | 0.25 | 0.57 | 339 | 1380 | 2429 |
| NPY1R | 0.0184 | 6.14E−07 | 0.13 | 0.23 | 0.57 | 190 | 813 | 1421 |
| CDH12 | 0.0125 | 4.16E−07 | 0.12 | 0.37 | 0.32 | 38 | 103 | 325 |
| ERBB4 | 0.0275 | 9.16E−07 | 0.11 | 0.24 | 0.46 | 40 | 169 | 369 |
| KLRG2 | 0.0306 | 1.02E−06 | 0.10 | 0.20 | 0.51 | 203 | 995 | 1960 |
| PPARGC1A | 0.0248 | 8.27E−07 | 0.09 | 0.25 | 0.34 | 25 | 97 | 285 |
| RAB3B | 0.0181 | 6.04E−07 | 0.05 | 0.20 | 0.26 | 69 | 342 | 1337 |

TABLE 5
Pathways enriched in eMF compared with NS

| Pathway | P-value |
|---|---|
| The Co-Stimulatory Signal During T-cell Activation | 9.5E−08 |
| CTL mediated immune response against target cells | 1.2E−04 |
| Lck and Fyn tyrosine kinases in initiation of TCR Activation | 4.2E−04 |
| Th1/Th2 Differentiation | 5.2E−04 |
| T Helper Cell Surface Molecules | 6.1E−04 |
| T Cytotoxic Cell Surface Molecules | 6.1E−04 |
| IL-2 Receptor Beta Chain in T cell Activation | 8.8E−04 |
| Activation of Csk by cAMP-dependent Protein Kinase Inhibits | 2.6E−03 |
| Signaling through the T Cell Receptor | |
| IL 2 signaling pathway | 5.8E−03 |
| B Lymphocyte Cell Surface Molecules | 6.0E−03 |
| Caspase Cascade in Apoptosis | 9.5E−03 |
| D4-GDI Signaling Pathway | 9.9E−03 |
| NO2-dependent IL 12 Pathway in NK cells | 9.9E−03 |
| IL-7 Signal Transduction | 1.2E−02 |
| IFN gamma signaling pathway | 1.8E−02 |
| T Cell Receptor Signaling Pathway | 3.2E−02 |

# The genes in Table 4 were analyzed using Database for Annotation, Visualization and Integrated Discovery (DAVID) Bioinformatics Resources 6.7 (Huang da et al., 2009a, b) See text for details.
eMF: early mycosis fungoides.
NS: normal skin.

### Example 2

### TOX as a Diagnostic and Prognostic Biomarker for T Cell Malignancy

TOX was further investigated as a biomarker for the diagnosis and prognosis of T Cell malignancy as set out below.

Levels of TOX mRNA in skin samples from subjects diagnosed with mycosis fungoides were compared to levels of TOX mRNA from subjects with benign inflammatory dermatoses or normal skin. As shown in FIG. 5, levels of TOX expression were observed to be significantly higher in subjects with mycosis fungoides compared to subject with benign inflammatory dermatoses or normal skin.

Subjects with mycosis fungoides were then classified according to disease stage. The levels of TOX in samples from subjects with stage I, stage II, stage III or stage IV mycosis fungoides were compared along with biopsy samples from subjects with benign inflammatory dermatoses, chronic dermatitis, pityriasis rubra pilaris, or normal skin. As shown in FIG. 6, the expression of TOX increases with disease progression from stage I to stage IV.

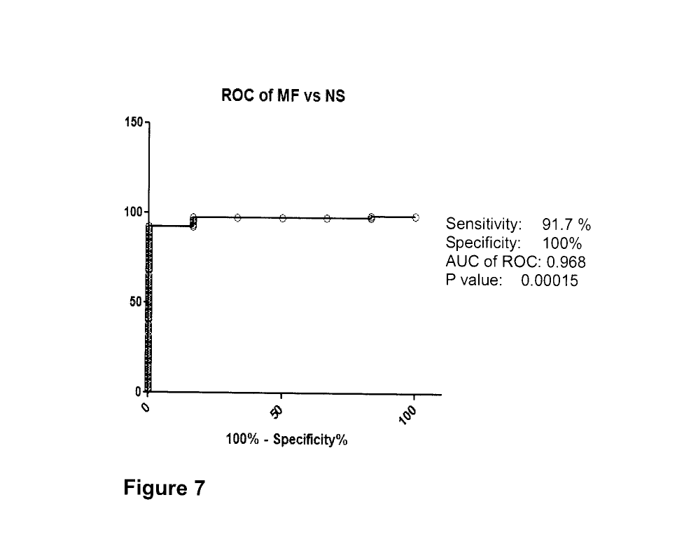

The utility of TOX as a biomarker for T cell malignancy was then investigated using Receiver Operator Characteristic (ROC) analysis. As shown in FIGS. 7 to 9, classifying subjects according to levels of TOX expression is useful for the diagnosis and prognosis of mycosis fungoides including in subjects with early stage patch or plaque disease.

TOX was also investigated as a biomarker in a population of patients with Sezary syndrome. As shown in FIG. 11, levels of TOX mRNA were higher is subjects with Sezary syndrome relative to levels in samples from subjects with psoriasis, rosacea, vitilligo and/or normal skin.

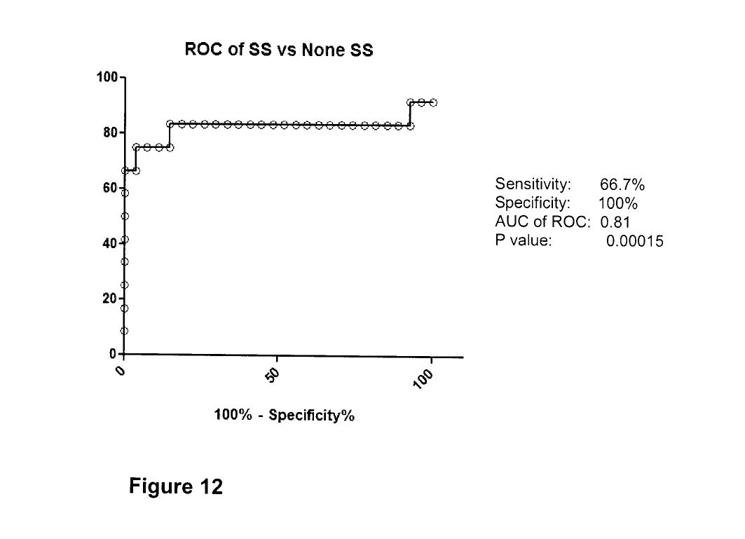

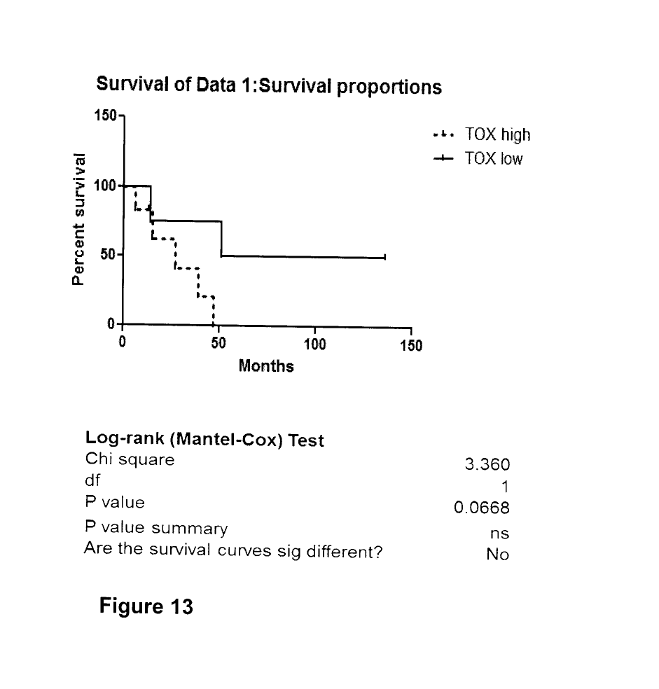

ROC analysis of TOX mRNA levels indicated that TOX is a statistically significant marker for Sezary syndrome with a sensitivity of 66.7% at a specificity of 100% (FIG. 12). Furthermore, TOX appears to be useful as a prognostic marker for predicting 5-year mortality in patients with Sezary syndrome (FIG. 13).

Western blots of protein preparations from cell lines from subjects with various forms of T cell malignancies were tested from the expression of TOX. As shown in FIG. 14, cells from peripheral T cell lymphoma (Jurkat) and chronic lymphoblastic leukemia (CCL119) express increased levels of TOX, similar to CTCL cells (patient derived and established cell lines) as compared to benign CD4+ T cells from healthy individuals.

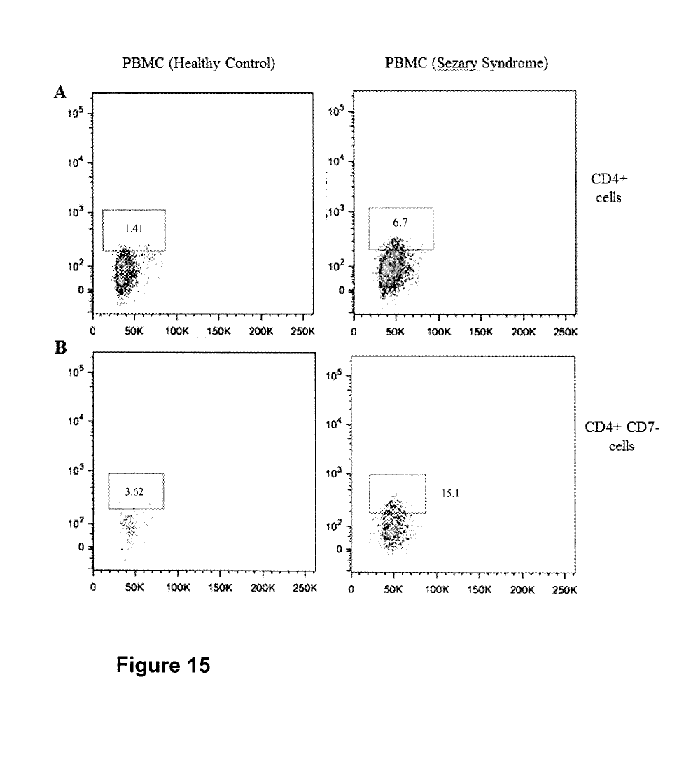

Fluorescence Activated Cell Sorting (FACS) was used to investigate the expression of TOX as well as CD7 in peripheral blood mononuclear cells (PBMCs) from a healthy control as well as from a patient with Sezary syndrome. The absence of CD7 expression is a molecular marker for CTCL. As shown in FIG. 15, TOX+ cells represented a higher proportion in PBMCs in the sample from the patient with Sezary syndrome relative to normal controls. Furthermore, TOX+ cells were enriched in the CD4+CD7− population.

While the present disclosure has been described with reference to what are presently considered to be the preferred examples, it is to be understood that the disclosure is not limited to the disclosed examples. To the contrary, the disclosure is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

### REFERENCES

Arai, E., I. Katayama, et al. (1991). “Mycosis fungoides and Sezary syndrome in Japan. Clinicopathologic study of 107 autopsy cases.” Pathol Res Pract 187(4): 451-457.
Asadullah, K., A. Haeussler-Quade, et al. (2000). “IL-15 and IL-16 overexpression in cutaneous T-cell lymphomas: stage-dependent increase in mycosis fungoides progression.” Exp Dermatol 9(4): 248-251.
Ashburner, M., C. A. Ball, et al. (2000). “Gene ontology: tool for the unification of biology. The Gene Ontology Consortium.” Nat Genet 25(1): 25-29.
Bustin, M. (1999). “Regulation of DNA-dependent activities by the functional motifs of the high-mobility-group chromosomal proteins.” Mol Cell Biol 19(8): 5237-5246.
Dai, D. L., N. Makretsov, et al. (2003). “Increased expression of integrin-linked kinase is correlated with melanoma progression and poor patient survival.” Clin Cancer Res 9(12): 4409-4414.
De Panfilis, G. (2002). “‘Activation-induced cell death’: a special program able to preserve the homeostasis of the skin?” Exp Dermatol 11(1): 1-11.
Doherty, S. D., X. Ni, et al. (2006). “Abnormal expression of interleukin-23 in mycosis fungoides/Sezary syndrome lesions.” Arch Dermatol Res 298(7): 353-356.
Fargnoli, M. C., R. L. Edelson, et al. (1997). “Diminished TCR signaling in cutaneous T cell lymphoma is associated with decreased activities of Zap70, Syk and membrane-associated Csk.” Leukemia 11(8): 1338-1346.
Ferrara, G., A. Di Blasi, et al. (2008). “Regarding the algorithm for the diagnosis of early mycosis fungoides proposed by the International Society for Cutaneous Lymphomas: suggestions from routine histopathology practice.” J Cutan Pathol 35(6): 549-553. Furmanczyk, P. S., G. M. Wolgamot, et al. (2010). “Diagnosis of mycosis fungoides with different algorithmic approaches.” J Cutan Pathol 37(1): 8-14.
Gambichler, T., M. Skrygan, et al. (2007). “Expression of human beta-defensins in patients with mycosis fungoides.” Arch Dermatol Res 299(4): 221-224.
Huang da, W., B. T. Sherman, et al. (2009a). “Bioinformatics enrichment tools: paths toward the comprehensive functional analysis of large gene lists.” Nucleic Acids Res 37(1): 1-13.
Huang da, W., B. T. Sherman, et al. (2009b). “Systematic and integrative analysis of large gene lists using DAVID bioinformatics resources.” Nat Protoc 4(1): 44-57.
Klemke, C. D., D. Brenner, et al. (2009). “Lack of T-cell receptor-induced signaling is crucial for CD95 ligand up-regulation and protects cutaneous T-cell lymphoma cells from activation-induced cell death.” Cancer Res 69(10): 4175-4183.
Leroy, S., S. Dubois, et al. (2001). “Interleukin-15 expression in cutaneous T-cell lymphoma (mycosis fungoides and Sezary syndrome).” Br J Dermatol 144(5): 1016-1023.
Litvinov, I. V., D. A. Jones, et al. (2010). “Transcriptional profiles predict disease outcome in patients with cutaneous T-cell lymphoma.” Clin Cancer Res 16(7): 2106-2114.
Lu, D., M. Duvic, et al. (2001). “The T-cell chemokine receptor CXCR3 is expressed highly in low-grade mycosis fungoides.” Am J Clin Pathol 115(3): 413-421.
Massone, C., K. Kodama, et al. (2005). “Histopathologic features of early (patch) lesions of mycosis fungoides: a morphologic study on 745 biopsy specimens from 427 patients.” Am J Surg Pathol 29(4): 550-560.
Olsen, E., E. Vonderheid, et al. (2007). “Revisions to the staging and classification of mycosis fungoides and Sezary syndrome: a proposal of the International Society for Cutaneous Lymphomas (ISCL) and the cutaneous lymphoma task force of the European Organization of Research and Treatment of Cancer (EORTC).” Blood 110(6): 1713-1722.
Pimpinelli, N., E. A. Olsen, et al. (2005). “Defining early mycosis fungoides.” J Am Acad Dermatol 53(6): 1053-1063.
Shin, J., S. Monti, et al. (2007). “Lesional gene expression profiling in cutaneous T-cell lymphoma reveals natural clusters associated with disease outcome.” Blood 110(8): 3015-3027.
Su, M. W., I. Dorocicz, et al. (2003). “Aberrant expression of T-plastin in Sezary cells.” Cancer Res 63(21): 7122-7127.
Tang, L., D. L. Dai, et al. (2006). “Aberrant expression of collagen triple helix repeat containing 1 in human solid cancers.” Clin Cancer Res 12(12): 3716-3722.
Tang, L., M. Su, et al. (2008). “Endothelin-3 is produced by metastatic melanoma cells and promotes melanoma cell survival.” J Cutan Med Surg 12(2): 64-70.
Thomas, J. O. and A. A. Travers (2001). “HMG1 and 2, and related ‘architectural’ DNA-binding proteins.” Trends Biochem Sci 26(3): 167-174.
Tracey, L., R. Villuendas, et al. (2003). “Mycosis fungoides shows concurrent deregulation of multiple genes involved in the TNF signaling pathway: an expression profile study.” Blood 102(3): 1042-1050.
Wang, Y., H. Jiang, et al. (2010). “Alpha 1 antichymotrypsin is aberrantly expressed during melanoma progression and predicts poor survival for patients with metastatic melanoma.” Pigment Cell Melanoma Res 23(4): 575-578.
Wang, Y., M. Su, et al. (2011). “Deficiency of SATB1 expression in Sezary cells causes apoptosis resistance by regulating FasL/CD95L transcription.” Blood.
Wang, Y., M. Su, et al. (2011). “Deficiency of SATB1 expression in Sezary cells causes apoptosis resistance by regulating FasL/CD95L transcription.” Blood 117(14): 3826-3835.
Wilkinson, B., J. Y. Chen, et al. (2002). “TOX: an HMG box protein implicated in the regulation of thymocyte selection.” Nat Immunol 3(3): 272-280.
Zhou, Y., D. L. Dai, et al. (2005). “Osteopontin expression correlates with melanoma invasion.” J Invest Dermatol 124(5): 1044-1052.

1. A method of screening for, diagnosing or detecting T cell malignancy in a subject, the method comprising:
(a) determining a level of TOX in a sample from the subject; and
(b) comparing the level of TOX in sample to a control level, wherein an increased level of TOX in the sample relative to the control level indicates that the subject has T cell malignancy
wherein the T-cell malignancy is Cutaneous T-cell Lymphoma (CTCL), peripheral T-cell lymphoma or T cell leukemia.

2. The method of claim 1, further comprising determining a level of one or more biomarkers listed in Table 2 in the sample from the subject.

3. (canceled)

4. The method of claim 1, wherein the CTCL is Mycosis Fungoides (MF) or Sezary Syndrome

5. The method of claim 4, wherein the MF is early stage Mycosis Fungoides (eMF).

6. (canceled)

7. The method of claim 1, wherein the sample is a tissue sample or a blood sample.

8. (canceled)

9. (canceled)

10. The method of claim 1, wherein determining the level of TOX in the sample from the subject comprises testing the sample for the expression of TOX.

11. The method of claim 10, wherein testing the sample for the expression of TOX comprises contacting the sample with a detection agent that selectively binds to a nucleic acid molecule that codes for the TOX protein.

12. (canceled)

13. The method according to claim 10, wherein testing the sample for the expression of TOX comprises contacting the sample with a detection agent that selectively binds to a nucleic acid molecule that codes for the TOX protein.

14. (canceled)

15. (canceled)

16. The method of claim 1, wherein the control level is representative of the level of TOX in one or more samples of normal skin.

17. (canceled)

18. The method claim 1, wherein the control level is representative of a level of TOX in a sample from the subject taken at an earlier time point.

19. (canceled)

20. The method of claim 1, further comprising providing a prognosis for the subject with T-cell malignancy wherein the magnitude of the level of TOX in the sample from the subject relative to the control level is indicative of the severity of disease.

21. The method of claim 20, wherein the control level is representative of the level of TOX in one or more samples from subjects with stage I, stage II, stage III or stage IV T-cell malignancy, optionally stage I, stage II, stage III or stage IV cutaneous T cell lymphoma.

22. A method of monitoring T cell malignancy in a subject comprising:
(a) determining a level of TOX in a sample from the subject at a first time point;
(b) determining a level of TOX in a sample from the subject at a second time point and comparing the level of TOX in the sample at the first time point with the level of TOX in the sample at the second time point,
wherein an increase in the level of TOX is indicative of an increase in severity of disease and a decrease in the level of TOX is indicative of a decrease in severity of disease.

23. (canceled)

24. The method of claim 22, wherein the T cell malignancy is cutaneous T Cell lymphoma (CTCL), peripheral T cell lymphoma or T cell leukemia.

24.-26. (canceled)

27. The method of claim 22, wherein determining a level of TOX in the sample comprises testing the sample for the expression of TOX.

28. The method of claim 22, wherein the subject is undergoing treatment for T cell malignancy and the method is used to monitor a response of the subject to the treatment.

29. A method of providing a prognosis for a subject with T cell malignancy comprising:
(a) determining a level of TOX in a sample from the subject; and
(b) comparing the level of TOX in the sample to a control level,
wherein the control level is representative of a level of TOX in one or more samples from subjects without T cell malignancy, and the magnitude of the level of TOX in the sample relative to the control level is indicative of the severity of the disease.

30. (canceled)

31. The method of claim 29, wherein the control level is representative of a level of TOX in one or more samples from subjects with stage I, stage II, stage III or stage IV T cell malignancy.

32. The method of claim 29, wherein the T cell malignancy is cutaneous T cell Lymphoma (CTCL), peripheral T cell lymphoma or T cell leukemia.

33. The method of claim 29, wherein the prognosis is the likelihood of the subject progressing to a least one numerical grade higher of T cell malignancy.

34. The method of claim 29, wherein the prognosis is the likelihood of mortality from the disease.

35. The method of claim 29, wherein determining a level of TOX in the sample comprises testing the sample for the expression of TOX.

36.-38. (canceled)

39. A kit comprising (i) reagents for conducting a method according to claim 1 and (ii) instructions for use, wherein the reagents comprise a detection agent specific for TOX.

40.-43. (canceled)

44. The method of claim 1, further comprising treating a subject identified as having the T-cell malignancy with an anticancer therapy or antineoplastic agent.
